(19) United States
(12) Patent Application Publication
(10) Pub. No.: US 2024/0252494 A1
(43) Pub. Date: Aug. 1, 2024

(54) Mechanotransduction Disruption Mediation in Skin Grafting Methods and Compositions for Use in Practicing the Same

(71) Applicant: The Board of Trustees of the Leland Stanford Junior University, Standford, CA (US)

(72) Inventors: Geoffrey Gurtner, Stanford, CA (US); Kellen Chen, Stanford, CA (US); Dominic Henn, Stanford, CA (US)

(21) Appl. No.: 18/288,652

(22) PCT Filed: Jul. 25, 2022
(86) PCT No.: PCT/US2022/038189

§ 371 Date: Oct. 27, 2023

Related U.S. Application Data
(60) Provisional application No. 63/227,811, filed on Jul. 30, 2021.
(60) Provisional application No. 63/340,145, filed on May 10, 2022.

(51) Int. Cl. A61K 31/506; A61B 17/322; A61L 26/00; A61L 27/36; A61L 27/60; A61P 17/02

(52) U.S. Cl. CPC A61K 31/506; A61B 17/322; A61L 26/0023; A61L 26/0066; A61L 26/008; A61L 27/362; A61L 27/60; A61P 17/02

(57) ABSTRACT

Skin graft methods are provided. Aspects of the methods include applying a skin graft to a wound in combination with a mechanotransduction blocker, such as a pharmacological mechanotransduction blocker, e.g., a focal adhesion kinase inhibitor. Also provided are compositions and kits for use practicing methods of the invention.

### CROSS-REFERENCE TO RELATED APPLICATIONS

Pursuant to 35 U.S.C. § 119 (e), this application claims priority to the filing date of U.S. Provisional Patent Application Ser. No. 63/227,811 filed on Jul. 30, 2021; and U.S. Provisional Patent Application Ser. No. 63/340,145 filed on May 10, 2022; the disclosure of which applications is herein incorporated by reference.

### ACKNOWLEDGEMENT OF GOVERNMENT RIGHTS

This invention was made with Government support under NIH Grant No. DE026914 awarded by the National Institute of Health. The Government has certain rights in the invention.

### INTRODUCTION

In humans and other large mammals, injuries typically result in scar formation, characterized by excessive fibrosis and loss of function (1-3). As the body's most superficial organ, the skin is usually the first line of defense against external traumatic forces, making it particularly susceptible to injury and subsequent hypertrophic scar formation and contracture. Cutaneous injuries may have multiple etiologies, but burns are among the most devastating and represent a major global public health burden (1, 4), especially in middle- and low-income countries (5). There are at least 40,000 annual cases of hospitalization related to burn injuries, and each of the 128 U.S. burn centers receives an average of 200 annual admissions for burn and burn-related skin disorders (6). Burn injuries restricted to the skin and/or subcutaneous fat are usually reconstructed using autologous skin transplantation with full-thickness or split-thickness skin grafts (STSGs) (7).

In the clinic, deep burns and other deep, large surface area injuries are almost never allowed to heal on their own. Instead, STSGs play a critical role, given their low donor site morbidity, promotion of a conducive healing environment, and ability to cover relatively large areas using graft meshing techniques. Although skin grafts serve an important function in rapidly restoring the barrier function of the skin to prevent infection and reduce mortality, secondary contraction during graft healing results in hypertrophic scar (HTS) formation (8). Healed skin grafts are also characterized by increased fragility, abnormal pigmentation, and poor texture, compared to unwounded skin (9). Revision rates following skin grafting have been reported to be as high as 20-30% (10), with younger patients sometimes requiring multiple rounds of skin grafting as they grow, presenting additional challenges in donor site availability (11, 12). Over time, skin graft contractures can form and are commonly addressed with contracture release and a new STSG, potentially restarting the vicious cycle of contracture. Unfortunately, there are no FDA (Food and Drug Administration)-approved pharmacologic therapies currently available for patients with cutaneous injuries to prevent debilitating fibrotic scar formation, contractures, and other functional complications following skin grafting (7, 13, 14).

Several recent studies have uncovered how upregulation of mechanical signaling can drive fibro-proliferative scarring and the development of fibrosis after injury in mice (15-21). Our group has identified the critical importance of mechanotransduction pathways in the skin and has shown that its inhibition can successfully attenuate scar formation and improve open wound healing (22-27). However, none of these studies have explored in detail the primary signal pathways that drive healing after skin grafting or skin transplantation. Additionally, mice are loose skinned animals, healing predominantly through contraction with less than 10% of the scarring that occurs in humans (28, 29). In contrast, humans and pigs are several orders of magnitude larger and are tight-skinned animals that heal through re-epithelialization over granulation tissue, resulting in considerably more scarring (27). Unfortunately, these differences affect the ability to translate discoveries from mice to humans.

The molecular and cellular mechanisms that underlie dermal remodeling and fibrosis after STSG in large animals remain incompletely understood (30, 31). Single-cell RNA sequencing (scRNA-seq) technologies have recently revolutionized how cells can be analyzed transcriptionally to elucidate the pathophysiology of diseases (32-34).

### SUMMARY

The present disclosure provides a method for treating a wound of a subject, the method comprising applying a skin graft to the wound in combination with a mechanotransduction blocker to treat the wound of the subject.

In some cases, the present disclosure provides a method of reducing scar formation after applying a skin graft to a treatment site of a human subject, the method comprising applying the skin graft to the treatment site; delivering a focal adhesion kinase inhibitor to the skin graft; and reducing scar formation at the treatment site.

Pharmaceutical compositions and kits for practicing the subject methods are also provided.

### BRIEF DESCRIPTION OF THE FIGURES

FIG. 1A-1E. Development of translational porcine model using clinically relevant methods. (A) Schematic of full-thickness excisional wounds on the pig dorsum and harvested skin grafts from a donor site. (B) Staged photos of full thickness wounds made using an electric bovie. (C) Photos of harvested and meshed skin grafts (0.01 in) at a 1:1.5 ratio. (D) Staged images showing the original unwounded skin, full thickness wound, skin graft secured with staples, and coverage with 3 layers of petrolatum gauze with a bolster dressing. (E) Photographic images of the graft at POD 0, 7, and 90.

FIG. 2A-2J. Cellular subpopulations in scar resulting from STSG are characterized by increased mechanotransduction and inflammatory signaling. (A) Left: Gross photography of unwounded skin (top) and STSG at 90 days (bottom). Scale bar=0.5 cm. Middle: Trichrome staining (scale bar=0.5 mm) and aSMA⁺ myofibroblast staining (scale bar=100 μm). Right: Picrosirius red staining of collagen fibers (scale bar=5 μm). (B) Quantification of dermal thickness, aSMA⁺ myofibroblasts, and collagen alignment using CurveAlign (109). Statistical comparison made using unpaired two-tailed t-tests (*p<0.05). All data represent mean±SEM of biological replicates (n=6 STSG per condition) (C) Schematic showing porcine cells isolated from STSG and unwounded skin tissue and processed for scRNA-seq. (D) UMAP embedding of all cells colored by cell type. (E) Number of differentially expressed genes (avg log fold change >0.5) for each cell type. (F) Feature plots of genes and (G) Genetrail3 Overrepresentation Analysis (ORA)-enriched pathways for myeloid cells. (H) UMAP embedding of fibroblasts. (I and J) Top feature and pathway plots for fibroblasts.

FIG. 3A-3G. Disruption of mechanotransduction in large animals accelerates STSG healing, attenuates fibrotic scar formation, reduces contracture, and improves biomechanical properties. (A and B) Schematic showing large area (25 cm²) full-thickness excisional wounds with STSG created on the lateral dorsum (left and right) of red Duroc pigs. STSG were either treated with standard bandage dressings, blank hydrogels (STSG+Blank; 30 STSG+B), or FAKI-releasing hydrogels (STSG+FAKI; STSG+F) (n=6 STSG per condition). All wounds were evaluated by gross photography. (C) Representative images tracking interstitial epithelialization, scar formation, and contracture over time. Scale bar=2.5 cm. (D) Scar contracture over time was measured and quantified. (E) Re-epithelialization of STSG at POD7 compared to unwounded skin. (F) Visual Analog Scale (VAS) scoring assessed by three blinded plastic surgeons from digital photographs. (G) STSG firmness assessed by deformation induced by cutometer (n=6 STSG per condition). Statistical comparisons made using one-way analysis of variance (ANOVA) with Tukey's multiple comparisons tests (*P<0.05). All data represent mean±SEM of biological replicates.

FIG. 4A-4F. FAKI-mediated inhibition of mechanotransduction on STSG of large animals reduces collagen and restores organization of collagen fiber networks. (A) Trichrome staining shows delineations of superficial and deep scar. Scale bar: 1 mm. (B and C) Picrosirius red staining of the three scar groups (STSG, STSG+Blank, STSG+FAKI) at POD90 was quantified and compared to unwounded skin for alignment (CurveAlign) and fiber length/width metrics (CT-Fire) (98, 109, 110) (n=6 STSG per condition). Scale bar: 10 μm. (D to F) Quantification of the different collagen fiber network characteristics across the four groups in the deep dermis. Statistical comparisons made using one-way analysis of variance (ANOVA) with Tukey's multiple comparisons tests (*P<0.05, *P<0.01, **P<0.001). All data represent mean±SEM of biological replicates.

FIG. 5A-5L. Mechanotransduction blockade causes an early (POD7) upregulation of anti-inflammatory pathways in myeloid cells. (A) Schematic of STSG and STSG+FAKI. (B) Representative photographs of early FAK inhibition on STSG. Scale Bar=1 cm. (C) Porcine cells shown in a UMAP embedding colored by STSG or STSG+FAKI. (D) UMAP embedding of cells colored by cell type. Dashed line shows myeloid cells of interest. (E) Number of differentially expressed genes between STSG and STSG+FAKI by cell type. (F) Violin plots of fibrotic genes expressed by fibroblasts. (G) UMAP embedding of myeloid cells colored by STSG or STSG+FAKI. (H) UMAP embeddings of myeloid cells colored by cell type with RNA velocity streams overlaid. (I) Violin plots of fibrotic or anti-inflammatory genes expressed by monocyte lineage cells. (J) Genetrail3 ORA pathway plots of myeloid cells. (K) Representative images of immunofluorescent staining of CXCL10 protein over time in porcine tissue. Scale bar=200 μm. HM (high magnitude) scale bar=50 μm. (L) Quantification of F4/80 and CXCL10 protein in porcine tissue over time at POD 7 (n=3 per condition), POD 14 (n=3 per condition), and POD 90 (n=6 per condition). Statistical comparisons made using two-way analysis of variance (ANOVA) with Tukey's multiple comparisons tests (*P<0.05, ***P<0.001). All data represent mean±SEM of biological replicates.

FIG. 6A-6F. Disruption of mechanotransduction shifts fibroblast transcriptional fates from pro-fibrotic to regenerative at late (POD90) time points. (A) Porcine cells were isolated from STSG treated with FAKI hydrogel (STSG+FAKI), control STSG, and unwounded skin. UMAP embedding of the fibroblasts colored by treatment group. RNA velocities shown as the main gene-averaged flow, visualized by velocity streamlines projected onto the UMAP embedding. (B) Six fibroblast lineages and terminal states as determined by CellRank (81). (C) Cells colored by latent time as computed by scVelo across all genes, quantifying overall differences in transcriptional dynamics between cells. (D) Heatmaps showing smoothed gene expression of the top genes with the highest correlation with regenerative (left, lineages 1,2) and fibrotic (right, lineages 3,4) fate probabilities, sorted according to latent time peaks. Center. Expression of APOE and ACAN genes across the six lineages. (E and F) Regenerative (left) versus fibrotic lineages (right) analyzed in more depth. Top: Expression of group-defining genes and key pathways projected onto the UMAP embedding. Bottom: Gene-specific RNA velocities. Dotted purple line represents estimated ‘steady-state’ ratio of unspliced:spliced mRNA. Positive velocities (higher abundance of unspliced mRNA than expected) indicate gene up-regulation.

FIG. 7A-7D. Time course of STSG healing across regenerative and fibrotic lineages. Protein confirmation performed using immunofluorescence staining of STSG and FAK-inhibited STSG porcine dermal tissue sections at POD 7 (n=3 per group), POD 14 (n=3 per group), and POD 90 (n=6 per group). Representative images for (A) CXCL14, (B) THBS4, (C) APOE, and (D) CD34. Scale bar=200 μm. HM (high magnitude) scale bar=50 μm. Statistical comparisons made using two-way analysis of variance (ANOVA) with Tukey's multiple comparisons tests (*P<0.05, ***P<0.001). All data represent mean±SEM of biological replicates.

FIG. 8A-8I. 3D organotypic scar system recapitulates two opposing trajectories of regeneration versus fibrosis in both human and porcine cells. (A) Schematic: Fibroblasts were isolated from human patient samples, cultured, and seeded into 3D collagen scaffolds. Collagen scaffolds were either subjected to no strain (NS, black), strain (blue), or strain and FAKI (Strain+FAKI, red). (n=3 per condition). Scale bar: 1 cm. (B and C) Immunofluorescent staining of top (B) fibrotic and (C) regenerative markers. Scale bar: 100 μm. Statistical comparisons made using one-way analysis of variance (ANOVA) with Tukey's multiple comparisons tests (*p<0.05, **p<0.01, ****p<0.0001). All data represent mean±SEM of biological replicates. (D) Schematic: Porcine fibroblasts were tested. (E) UMAP embedding of fibroblasts with velocity embedded streams, colored by group. (*) denotes root origin of differentiation. (F) UMAP embedding colored by latent time. (G) Heatmap of the top differentially expressed genes by group. (H and I) Regenerative (H) versus fibrotic lineages (I) observed in our 3D system. Expression of group-defining genes projected onto the UMAP embedding (top) or violin plots (bottom).

FIG. 9A-9E. Diverse cellular ecology observed in chronic porcine STSG and unwounded skin. (A) Cell-type defining genes to confirm our automated cell type annotations. (B) Representative proportions of each cell type between STSG and unwounded skin. (C, D) Heatmaps of differentially expressed genes by cell types. (E) Additional feature plots of genes and enriched pathways in fibroblasts.

FIG. 10A-10C. Hydrogel releases FAKI over time into the dermis. (A) Schematic of hydrogel delivery of FAKI to the STSG. (B) FAKI hydrogels within dialysis membranes show steady release of FAKI over time. (n=2 for blank; n=3 for FAKI). Statistical comparisons made using two-way analysis of variance (ANOVA) with Tukey's multiple comparisons tests (*P<0.05, **P<0.01, ***P<0.001). All data represent mean±SEM of biological replicates. (C) Penetration of FAKI into the dermis over time.

FIG. 11A-11C. Fiber analysis was performed on both the deep and superficial dermis. Additional images and quantification of picrosirius red-stained images was performed (n=6 per condition). Statistical comparisons were made using analysis of variance (ANOVA) with Tukey's multiple comparisons tests (*P<0.05, **P<0.01, ***P<0.001, ****P<0.0001). Data represent mean±SEM of biological replicates.

FIG. 12A-12C. Diverse cellular ecology was observed in early (POD7) STSG and STSG+FAKI. (A) Cell-type defining genes confirm our automated cell type annotations. (B, C) Violin plots of cluster-defining differentially expressed genes in (B) fibroblasts and (C) monocytes and macrophages. Box plots are overlaid to show the medians and interquartile ranges.

FIG. 13A-13F. CellRank and scVelo analysis of late (POD90) fibroblasts. (A) Initial and (B) terminal states identified by CellRank. (C) Velocity vectors shown for each individual cell. (D) Velocity length indicates increased transcriptional magnitudes across all genes. (E, F) Gene-resolved velocities for (E) ENPP1 and (F) ACTA2. The dotted line represents the estimated ‘steady-state’ ratio of unspliced to spliced mRNA abundance.

FIG. 14A-14D. Additional analysis of fibroblasts from late (POD90) STSG, STSG+FAKI, and unwounded skin. (A) Violin plots of cluster-defining differentially expressed genes. Box plots are overlaid to show the medians and interquartile ranges. (B) Heatmap of the top differentially expressed genes by treatment group. (C) Select feature plots illustrating gene expression. (D) GeneTrail feature UMAP plots of key pathways that differentiate between the groups.

FIG. 15. Protein confirmation of scRNA-seq observations in human patient samples. Protein confirmation performed using immunofluorescence staining of human hypertrophic scar (HTS) (n=6 samples) and unwounded skin (n=3 samples) collected from patient samples. Staining for THBS4, which contributes to excessive scar formation, or APOE, which contributes to regenerative, adipogenic dermal healing. Scale bar: 50 μm. Statistical comparisons were made using unpaired two-tailed t-tests (P<0.05, ****P<0.0001). All data represent mean±SEM of biological replicates.

FIG. 16. Immunofluorescent staining of αSMA in human collagen scaffolds. Scale bar: 100 μm. Statistical comparisons made using one-way analysis of variance (ANOVA) with Tukey's multiple comparisons tests (**P<0.01). All data represent mean±SEM of biological replicates.

FIG. 17. Violin plots of in vitro porcine scRNA-seq data with box plots overlaid to show the medians and interquartile ranges.

### DEFINITIONS

As used herein in its conventional sense, the term “fibroblast” refers to a cell responsible for synthesizing and organizing extracellular matrix. Two fibroblast lineages include Engrailed-1 lineage-negative fibroblasts (ENFs) and Engrailed-1 lineage-positive fibroblasts (EPFs). The EPF lineage includes all cells that express Engrailed-1 at any point during their development, and all progeny of those cells.

The term “fibrosis” as used herein in its conventional sense refers to the formation or development of excess fibrous connective tissue in an organ or tissue as a result of injury or inflammation of a part or interference with its blood supply. It can be a consequence of the normal healing response that leads to a scar, an abnormal reactive process or no known or understood cause.

As used herein in its conventional sense, the term “scar” refers to a fibrous tissue that replaces normal tissue destroyed by injury or disease. Damage to the outer layer of skin (the epidermis) is healed by rebuilding the tissue, and in these instances, scarring is slight or absent. When the thick layer of tissue beneath the skin's outer surface (i.e., the dermis) is damaged, however, rebuilding is more complicated. The body lays down collagen fibers (a protein which is naturally produced by the body) in a composition that is different from that found in uninjured skin, and this usually results in a noticeable scar. After the wound has healed, the scar continues to alter as new collagen is formed, existing collagen is enzymatically remodeled, and the blood vessels return to normal, allowing most scars to fade and improve in appearance over the two years following an injury. However, there permanently remains some visible evidence of the injury, and hair follicles and sweat and oil glands do not grow back. As used herein, the term “scar area” refers to the area of normal tissue that is destroyed by injury or disease and replaced by fibrous tissue.

Scars differ from normal skin in three key ways: (1) they are devoid of any dermal appendages (hair follicles, sweat glands, etc.); (2) their collagen structure is fundamentally different, with dense, parallel fibers rather than the “basketweave” pattern that lends normal skin its flexibility and strength; and (3) as a result of their inferior matrix structure, they are weaker than skin.

The term “scar-related gene” as used herein refers to a nucleic acid encoding a protein that is activated in response to scarring as part of the normal wound healing process. The term “scar-related gene product” as used herein refers to the protein that is expressed in response to scarring as part of the normal wound healing process.

Scar tissue consists mainly of disorganized collagenous extracellular matrix. This is produced by myofibroblasts, which differentiate from dermal fibroblasts in response to wounding, which causes a rise in the local concentration of Transforming Growth Factor-β, a secreted protein that exists in at least three isoforms called TGF-βI, TGF-β2 and TGF-β3 (referred to collectively as TGF-β). TGF-β is an important cytokine associated with fibrosis in many tissue types (Beanes, S. et al, Expert Reviews in Molecular Medicine, vol. 5, no. 8, pp. 1-22 (2003)). Types of scars are further described in, e.g., PCT Application No. WO 2014/040074, the disclosure of which is incorporated herein by reference in its entirety.

The term “skin” used herein in its conventional sense includes all surface tissues of the body and sub-surface structure thereat including, e.g., mucosal membranes and eye tissue as well as ordinary skin. The expression “skin” may include a wound zone itself. The re-approximation of skin over the surface of a wound has long been a primary sign of the completion of a significant portion of wound healing. This reclosure of the defect restores the protective function of the skin, which includes protection from bacteria, toxins, and mechanical forces, as well as providing the barrier to retain essential body fluids. The epidermis, which is composed of several layers beginning with the stratum corneum, is the outermost layer of the skin. The innermost skin layer is the deep dermis.

As used herein in its conventional sense, the term “dermal appendages” includes hair follicles, sebaceous and sweat glands, fingernails, and toenails.

As used herein, the term “dermal location” refers to a region of a skin of a subject having any size and area. The dermal location may encompass a portion of skin of a subject such as, e.g., the scalp. The dermal location may include one or more layers of skin including, e.g., the epidermis and the dermis. In some cases, the dermal location includes a wound.

As used herein in its conventional sense, the term “wound” includes any disruption and/or loss of normal tissue continuity in an internal or external body surface of a human or non-human animal body, e.g. resulting from a non-physiological process such as surgery or physical injury. The expression “wound” or “wound environment” used herein refers to any skin lesion capable of triggering a healing process which may potentially lead to scarring, and includes wounds created by injury, wounds created by burning, wounds created by disease and wounds created by surgical procedures. The wound may be present on any external or internal body surface and may be penetrating or non-penetrating. The methods herein described may be beneficial in treating problematic wounds on the skin's surface. Examples of wounds which may be treated in accordance with the method of the invention include both superficial and non-superficial wounds, e.g. abrasions, lacerations, wounds arising from thermal injuries (e.g. burns and those arising from any cryo-based treatment), and any wound resulting from surgery.

The term “wound healing” as used herein in its conventional sense refers to a regenerative process with the induction of a temporal and spatial healing program, including, but not limited to, the processes of inflammation, granulation, neovascularization, migration of fibroblast, endothelial and epithelial cells, extracellular matrix deposition, re-epithealization, and remodeling.

Hydrogel. Hydrogels useful in the methods of the invention maintain viability of entrapped cells for a period of time sufficient to enhance wound healing. Hydrogels are known and used in the art for wound healing. Typically hydrogels are, by weight, up to about 50%, up to about 55%, up to about 60%, up to about 65%, up to about 70%, up to about 75%, up to about 80%, up to about 85%, up to about 90% water, with the remaining weight comprising a suitable polymer, e.g. pullulan and collagen, glycosaminoglycan, acrylate, 2-hydroxymethyl methacrylate and ethylenedimethacrylate copolymer, carboxymethylcellulose, chitosan, gelatin, etc., or other suitable hydrophilic polymers as known in the art. Hydrogels can swell extensively without changing their gelatinous structure and are available for use as amorphous (without shape) gels and in various types of application systems, e.g. flat sheet hydrogels and non-woven dressings impregnated with amorphous hydrogel solution. Flat sheet (film) hydrogel dressings have a stable cross-linked macrostructure and therefore retain their physical form as they absorb fluid.

In some embodiments a cross-linked hydrogel film is fabricated using pullulan and collagen under conditions that provided for cross-linking and pore formation. Collagen is added to a mixture of pullulan, cross-linking agent and pore-forming agent (porogen), where the collagen is provided at a concentration of at least about 1%, and not more than about 12.5% relative to the dry weight of the pullulan. Collagen may be provided at a concentration of about 1%, about 2.5%, about 5%, about 7.5%, about 10%, usually at a concentration of from about 2.5% to about 10%, and may be from about 4% to about 6% relative to the dry weight of the pullulan. The collagen is typically a fibrous collagen, e.g. Type I, II, III, etc. Cross-linking agents of interest include sodium trimetaphosphate (STMP) or a combination of or a combination of sodium trimetaphosphate and sodium tripolyphosphate (STMP/STPP). The cross-linking agent can be included in a wt/wt ratio relative to the pullulan of from about 5:1 to about 1:5, and may be about 4:1, 3:1, 2:1, 1.75:1, 1.5:1, 1.25:1, 1:1, 1:1.25, 1:1.5, 1:1.75, 2:1, 3:1, 4:1, etc. Porogens of interest for in-gel crystallization include any suitable salt, e.g. KCl. The porogen can be included in a wt/wt ratio relative to the pullulan of from about 5:1 to about 1:5, and may be about 4:1, 3:1, 2:1, 1.75:1, 1.5:1, 1.25:1, 1:1, 1:1.25, 1:1.5, 1:1.75, 2:1, 3:1, 4:1, etc. The suspension of collagen, pullulan, cross-linker and porogen, in the absence of cells, is poured and compressed to form sheets. Preferred thickness is at least about 1 mm and not more than about 5 mm, usually not more than about 3 mm, and may be from about 1 to 2.5 mm, e.g. about 1.25, 1.5, 1.75, 2 mm thick. Pores are formed in the hydrogel through rapid desiccation of swollen hydrogels by phase inversion. Dehydration results in localized super-saturation and crystallization of the porogen. Pullulan and collagen are forced to organize around the crystals in an interconnected network, which results in reticular scaffold formation following KCl dissolution.

The films may be stored in a dried state, and are readily rehydrated in any suitable aqueous medium. The aqueous nature of hydrogel substrates provides an ideal environment for cellular growth and sustainability.

Mechanical features of the hydrogel include average pore size and scaffold porosity. Both variables vary with the concentration of collagen that is present in the hydrogel. For a hydrogel comprising 5% collagen, the average pore size will usually range from about 25 μm to about 50 μm, from about 30 μm to about 40 μm, and may be about 35 μm. For a hydrogel comprising 10% collagen the average pore size will usually range from about 10 μm to about 25 μm, from about 12 μm to about 18 μm, and may be about 15 m. One of skill in the art will readily determine suitable hydrogels at other collagen concentrations. The scaffold porosity will usually range from 5 about 50% to about 85%, and may range from about 70% to about 75%, and will decrease with increasing concentrations of collagen. Hydrogels lacking collagen do not display any birefringence with polarizing light microscopy, while the hydrogels comprising collagen are diffusely birefringent.

Pullulan. A polysaccharide produced by the fungus Aureobasidium pullulans. It is a linear homopolysaccharide consisting of alpha-(1-6) linked maltotriose units and exhibits water retention capabilities in a hydrogel state which makes it an ideal therapeutic vehicle for both cells and biomolecules. Additionally, pullulan contains multiple functional groups that permit cross-linking and delivery of genetic material and therapeutic cytokines. Furthermore, pullulan-based scaffolds have been shown to enhance both endothelial cell and smooth muscle cell behavior in vitro.

Collagen. As used herein the term “collagen” refers to compositions in which at least about 50%, at least about 60%, at least about 70%, at least about 80%, at least about 90%, at least about 95% or more of the protein present is collagen in a triple helical configuration. Collagens are widely found in vertebrate species, and have been sequenced for many different species. Due to the high degree of sequence similarity between species, collagen from different species can be used for biomedical purposes, e.g. between mammalian species. Typical commercial animal sources include the bovine Achilles tendon, calfskin and the bones of cattle. In some embodiments the collagen used in the preparation of the oriented thin film is Type I, Type II, or Type III collagen, and is derived from any convenient source, e.g. bovine, porcine, etc., usually a mammalian source.

Collagen has a triple-stranded rope-like coiled structure. The major collagen of skin, tendon, and bone is collagen I, containing 2 alpha-1 polypeptide chains and 1 alpha-2 chain. The collagen of cartilage contains only 1 type of polypeptide chain, alpha-1. The fetus also contains collagen of distinctive structure. The genes for types I, II, and III collagens, the interstitial collagens, exhibit an unusual and characteristic structure of a large number of relatively small exons (54 and 108 bp) at evolutionarily conserved positions along the length of the triple helical gly-X-Y portion.

Types of collagen include I (COL1A1, COL1A2); II (COL2A1); III (COL3A1); IV (COL4A1, COL4A2, COL4A3, COL4A4, COL4A5, COL4A6); V (COL5A1, COL5A2, COL5A3); VI (COL6A1, COL6A2, COL6A3); VII (COL7A1); VIII (COL8A1, COL8A2); IX (COL9A1, COL9A2, COL9A3); X (COL10A1); XI (COL11A1, COL11A2); XII (COL12A1); XIII (COL13A1); XIV (COL14A1); XV (COL15A1); XVI (COL16A1); XVII (COL17A1); XVIII (COL18A1); XIX (COL19A1); XX (COL20A1); XXI (COL21A1); XXII (COL22A1); XXIII (COL23A1); XXIV (COL24A1); XXV (COL25A1); XXVII (COL27A1); XXVIII (COL28A1). It will be understood by one of skill in the art that other collagens, including mammalian collagens, e.g. bovine, porcine, equine, etc. collagen, are equally suitable for the methods of the invention.

Focal adhesion kinase (FAK). FAK is a non-receptor cytoplasmic tyrosine kinase. FAK is one of the key mediators of skin mechanobiology and it is activated after cutaneous injury. Mechanical forces potentiate the activation of FAK through phosphorylation following injury of the skin. FAK contributes to cell signaling through its linking of mechanical stress from the ECM to the cytoplasmic cytoskeleton, activating inflammatory pathways. Fibroblasts are recruited to the wound by inflammatory signaling, where their secretion of profibrotic cytokines brings about increased collagen synthesis. Various pathologies that are associated with poor wound healing have been shown to have atypical levels of FAK. Mechanical force regulates pathologic scarring through inflammatory FAK-ERK-MCP1 pathways, and molecular strategies targeting focal adhesion kinase (FAK) can effectively uncouple mechanical force from fibrosis.

Wound dressing. films of the invention find use as a wound dressing, or artificial skin, by providing an improved substrate that minimizes scarring. An effective bioactive wound dressing can facilitate the repair of wounds that may require restoration of both the epidermis and dermis. For example, a hydrogel thin film is placed onto, and accepted by, the debrided wound of the recipient and provide a means for the permanent re-establishment of the dermal and epidermal components of skin. The graft suppresses the formation of granulation tissue which causes scarring.

Additional criteria for biologically active wound dressings include: rapid adherence to the wound soon after placement; proper vapor transmission to control evaporative fluid loss from the wound and to avoid the collection of exudate between the wound and the dressing material. Skin substitutes should act as barrier to microorganisms, limit the growth of microorganisms already present in the wound, be flexible, durable and resistant to tearing. The substitute should exhibit tissue compatibility, that is, it should not provoke inflammation or foreign body reaction in the wound which may lead to the formation of granulation tissue. An inner surface structure of a hydrogel thin film is provided that permits ingrowth of fibro-vascular tissue. An outer surface structure may be provided to minimize fluid transmission and promote epithelialization.

Typical bioabsorbable materials for use in the fabrication of porous wound dressings, skin substitutes and the like, include synthetic bioabsorbable polymers such as polylactic acid or polyglycolic acid, and also, biopolymers such as the structural proteins and polysaccharides. The finished dressing prior to cell seeding is packaged and preferably radiation sterilized. Such biologically active products can be used in many different applications that require the regeneration of dermal tissues, including the repair of injured skin and difficult-to-heal wounds, such as burn wounds, venous stasis ulcers, diabetic ulcers, etc.

Split-thickness grafts. Split-thickness or partial thickness skin grafts are usually used; for these grafts, a thin layer of epidermis and some dermis are excised and placed on the recipient site. Such grafts are typically used for burns but may also be used to accelerate healing of small wounds. Because a significant amount of dermal elements remain at the donor site, the site eventually heals and can be harvested again.

Full-thickness grafts. Full-thickness skin grafts are composed of epidermis and dermis and provide better appearance and function than split-thickness grafts. However, because the donor site will not heal primarily, it must be a loose area of redundant skin (eg, abdominal or thoracic wall, sometimes scalp) so that the site can be sutured closed. Thus, full-thickness grafting is usually reserved for cosmetically sensitive areas (eg, face) or areas requiring a thicker, more protective skin layer (eg, hands). Because full-thickness grafts are thicker and more vascular, they do not have quite as high a survival rate as split-thickness grafts. Split-thickness skin grafts may be classified as thin (0.15-0.25 mm) intermediate (0.3-0.4 mm) or thick (0.5-0.6 mm).

Composite grafts. Composite skin grafts include two or more different types of tissues. Most commonly, composite skin grafts have cartilage with or without subcutaneous tissue and the overlying skin. Because they offer support and structure composite grafts are often used to repair full-thickness defects of the nasal ala and helical rim.

The term “autograft” as used herein refers to a skin graft where the graft tissues is from the individual or subjects own body.

The term “allograft” as used herein refers to a skin graft where the graft tissues is from a different individual or subject other than the subject that is receiving treatment of a wound.

The term “xenograft” as used herein refers to a skin graft where the graft tissue is from an individual that is a different species or is synthetic graft tissue relative to the subject that is receiving the graft tissue. For example, a xenograft may be wherein a human is treated for a wound with a porcine skin graft.

### DETAILED DESCRIPTION

Skin graft methods are provided. Aspects of the methods include applying a skin graft to a wound in combination with a mechanotransduction blocker, such as a pharmacological mechanotransduction blocker, e.g., a focal adhesion kinase inhibitor. Also provided are pharmaceutical compositions and kits for use practicing methods of the invention.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

While the apparatus and method has or will be described for the sake of grammatical fluidity with functional explanations, it is to be expressly understood that the claims, unless expressly formulated under 35 U.S.C. § 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. § 112 are to be accorded full statutory equivalents under 35 U.S.C. § 112.

### Methods for Treating a Wound of a Subject

As summarized above, methods are provided for treating a wound of a subject, the methods include applying a skin graft to the wound in combination with a mechanotransduction blocker to treat the wound of the subject. The wound may be any wound of a subject in need of treatment. Wounds that receive benefit from the methods described herein include, without limitation, partial- and full-thickness wounds, ulcers, including pressure ulcers, diabetic ulcers (e.g., diabetic foot ulcers), venous ulcers, lower leg ulcer, etc.; burns (second and third degree burns) including scalds, chemical burns, thermal burns such as flame burns and flash burns, ultraviolet burns, contact burns, radiation burns, electrical burns, etc.; gangrene; skin tears or lacerations, such as made by knives, etc.; a incisions such as made by knives, nails, sharp glass, razors, etc.; avuls; amputations; surgical wounds; failing or compromised skin/muscle grafts or flaps; bites; slash wounds, i.e., a wound where the length is greater than the depth; bruises; and the like, or a combination of one or more of the above.

Subjects of the present disclosure may be any subject in need of treatment of a wound. In some embodiments, the subject is a mammal. Non-limiting examples of mammals that would benefit from the methods disclosed herein include, without limitation, canines; felines; equines; bovines; porcines; ovines; rodentia, such as mice or rats, etc. and primates, e.g., non-human primates, humans, etc. In a preferred embodiment, the mammal is a human.

The methods of the present disclosure involve applying a skin graft to the wound. The skin graft may be any skin graft deemed useful in the treatment of the wound of the subject. Skin grafts that find use in the present disclosure include, without limitation, full-thickness grafts, partial-thickness grafts, composite grafts, etc. The skin graft may be an autograft, an allograft or a xenograft. In some embodiments, the skin graft is applied before application of the mechanotransduction blocker.

In addition to applying a skin graft, the methods also involve applying a mechanotransuction blocker. Mechanotransduction blocker that find use in the present disclosure are any inhibitors that impair mechanotransduction signaling pathways. Non-limiting examples of mechanotranduction blockers include, without limitation, integrin inhibitors, focal adhesion kinase (FAK) inhibitors, Talin inhibitors, Vinculin inhibitors, Paxillin inhibitors, Zyxin inhibitors, VASP inhibitors, p130^{cas} inhibitors, etc. In some embodiments, the mechanotransduction inhibitor is a focal adhesion kinase (FAK) inhibitor. Non-limiting examples of FAK inhibitors include, without limitation, PF-56227, PF-573228, TAE226 (NVP-TAE226), BI-4464, GSK2256098, PF-431396, PND-1186 (VS-4718), Y15, Defactinib (VS-6063), Solanesol (Nonaisoprenol), etc. In addition to the FAK inhibitors disclosed above, other types of inhibitors may be used. For example, other types of FAK inhibitors include, without limitation, siRNA, anti-sense oligonucleotides (ASO). CRISPR-mediated knockout or knockdown of FAK, etc. In some embodiments, the mechanotransduction blocker is a pharmacological mechanotransduction blocker.

The mechanotransduction blockers of the present disclosure may be applied in any way deemed useful. In some embodiments, the mechanotransduction blocker is applied systemically. In some embodiments, the mechanotransduction blocker is applied locally at the site of the skin graft. When the mechanotransduction blocker is applied locally, the mechanotransduction blocker may be applied in a sustained release formulation. In some embodiments, the sustained release formulation includes a gel formulation. In some embodiments, the gel formulation includes a hydrogel. In some embodiments, the hydrogel includes a carbohydrate based hydrogel, e.g., a biodegradable pullulan-based hydrogel. Pullulan-based hydrogels are known in the art and have been described in Wong et al. (Tissue Eng Part A. 2011 March; 17(5-6):631-44) and Wong et al. (Macromol Biosci. 2011 Nov. 10; 11(11):1458-66), each herein specifically incorporated by reference.

The methods disclosed herein provide a number of benefits to wound healing relative to other methods, i.e. a skin graft in the absence of a mechanotransduction blocker. For instance, the methods may promote the healing of the wound, reduce fibrosis, reduce contracture, mitigate scar formation, restore collagen architecture, or improve graft biomechanical properties.

Embodiments of the methods disclosed herein reduce the amount of contracture occurring following the skin graft. Contracture is a measure of the change in scar area relative to the area of the skin graft. Contracture is the result of scar formation pulling on the edges of the skin surrounding the scar cause strain. The methods disclosed herein result in a range of reductions in contracture. For instance, contracture may be reduced by 1%, 2%, 3%, 4%, 5%, 6%, 7%, 8%, 9% 10%, 12%, 14%, 16%, 18%, 20%, 25%, 30%, 35%, 40%, 45%, 50% or greater than a 50% reduction in contracture relative to skin grafts in the absence of a mechanotransduction blocker.

Embodiments of the methods disclosed herein promote wound healing following a skin graft. In some embodiments, the promotion of wound healing is an increase in re-epithelialization. In embodiments where the promotion of wound healing results in an increase in re-epithelialization, a range of increases in re-epithelialization may occur. For example, re-epithelization may be increase by 1%, 2%, 3%, 4%, 5%, 6%, 7%, 8%, 9% 10%, 12%, 14%, 16%, 18%, 20%, 25%, 30%, 35%, 40%, 45%, 50% or greater than a 50% increase in re-epithelialization relative to skin grafts in the absence of a mechanotransduction blocker.

Embodiments of the methods disclosed herein improve skin graft biomechanical properties. In some embodiments, the improvement of skin graft biomechanical properties is a decrease in the firmness and an increase in elasticity of the skin graft as measured by the vertical deformation of the graft. In embodiments where the improvement of skin graft biomechanical properties is a decrease in the firmness and an increase in elasticity of the skin graft as measured by the vertical deformation of the graft, a range of increases in deformation may occur. For example, deformation may increase by 1%, 2%, 3%, 4%, 5%, 6%, 7%, 8%, 9% 10%, 12%, 14%, 16%, 18%, 20%, 25%, 30%, 35%, 40%, 45%, 50% or greater than a 50% increase in deformation of skin grafts relative to skin grafts in the absence of a mechanotransduction blocker.

Embodiments of the methods disclosed herein restore collagen architecture. In some embodiments, the restoration of collagen architecture is a decrease in the alignment and length of collagen fibers relative to skin grafts without a mechanotransduction blocker. Unwounded skin is generally characterized as having short and randomly aligned collagen. In embodiments where a restoration in collagen architecture is a decrease in the alignment of collagen fibers, a range of decreases in alignment of collagen fibers may occur. For instance, alignment of collagen fibers may decrease by 1%, 2%, 3%, 4%, 5%, 6%, 7%, 8%, 9% 10%, 12%, 14%, 16%, 18%, 20%, 25%, 30%, 35%, 40%, 45%, 50% or greater than a 50% decrease in alignment of collagen fibers of skin grafts relative to skin grafts in the absence of a mechanotransduction blocker. In embodiments where a restoration in collagen architecture is a decrease in the length of collagen fibers, a range of decreases in length of collagen fibers may occur. For instance, length of collagen fibers may decrease by 1%, 2%, 3%, 4%, 5%, 6%, 7%, 8%, 9% 10%, 12%, 14%, 16%, 18%, 20%, 22%, 24%, 26%, 28%, 30%, 32%, 40%, or greater than a 40% decrease in length of collagen fibers of skin grafts relative to skin grafts in the absence of a mechanotransduction blocker.

### Methods of Reducing Scar Formation

As summarized above, methods are provided for reducing scar formation after applying a skin graft to a treatment site of a human subject, the methods including applying the skin graft to the treatment site; delivering a focal adhesion kinase inhibitor to the skin graft to reduce scar formation at the treatment site.

The skin graft may be any skin graft deemed useful in the treatment of the wound of the subject. Skin grafts that find use in the present disclosure include, without limitation, full-thickness grafts, partial-thickness grafts, composite grafts, etc. The skin graft may be an autograft, an allograft or a xenograft.

A treatment site of the present disclosure may be any site on the skin that is in need of treatment. Treatment sites that find use in the present disclose include, without limitation, hand, palm, lower arm, upper arm, under arm, chest, abdomen, shoulders, upper back, lower back, neck, face, scalp, pelvis, groin, upper leg, lower leg, feet, etc.

FAK inhibitors that find use in the method disclosed herein are any FAK inhibitors that impair FAK based signaling. Non-limiting examples of FAK inhibitors include, without limitation, PF-56227, PF-573228, TAE226 (NVP-TAE226), BI-4464, GSK2256098, PF-431396, PND-1186 (VS-4718), Y15, Defactinib (VS-6063), Solanesol (Nonaisoprenol), etc. In addition to the FAK inhibitors disclosed above, other types of inhibitors may be used. For example, other types of FAK inhibitors include, without limitation, siRNA, anti-sense oligonucleotides (ASO). CRISPR-mediated knockout or knockdown of FAK, etc.

FAK inhibitors of the present disclosure may be delivered in a number of different ways. In some embodiments, the FAK inhibitor is delivered systemically. In some embodiments, the FAK inhibitor is applied locally at the treatment site. In some embodiments, the FAK inhibitor is applied in a sustained release formulation. In some embodiments, the sustained release formulation includes a gel formulation. In some embodiments, the gel formulation includes a hydrogel. In some embodiments, the hydrogel includes a biodegradable pullulan-based hydrogel.

Reduction of scar formation may present in a number of different ways. In some embodiments, the reduction in scar formation is reducing in the visual appearance of a scar. In some embodiments, the reduction in scar formation is a reduction in the contracture that occurs during and after scar formation. The methods disclosed herein result in a range of reductions in contracture. For instance, contracture may be reduced by 1%, 2%, 3%, 4%, 5%, 6%, 7%, 8%, 9% 10%, 12%, 14%, 16%, 18%, 20%, 25%, 30%, 35%, 40%, 45%, 50% or greater than a 50% reduction in contracture relative to skin grafts in the absence of a mechanotransduction blocker.

### Combination Therapy

For use in the subject methods, the mechanotransduction blocker(s), such as described above, may be administered in combination with other pharmaceutically active agents, including other agents that treat the underlying condition or a symptom of the condition, e.g., scarring. “In combination with” as used herein refers to uses where, for example, the first compound is administered during the entire course of administration of the second compound; where the first compound is administered for a period of time that is overlapping with the administration of the second compound, e.g. where administration of the first compound begins before the administration of the second compound and the administration of the first compound ends before the administration of the second compound ends; where the administration of the second compound begins before the administration of the first compound and the administration of the second compound ends before the administration of the first compound ends; where the administration of the first compound begins before administration of the second compound begins and the administration of the second compound ends before the administration of the first compound ends; where the administration of the second compound begins before administration of the first compound begins and the administration of the first compound ends before the administration of the second compound ends. As such, “in combination” can also refer to regimen involving administration of two or more compounds. “In combination with” as used herein also refers to administration of two or more compounds which may be administered in the same or different formulations, by the same of different routes, and in the same or different dosage form type.

Examples of other agents for use in combination therapy in embodiments of methods of the invention include, but are not limited to, YAP inhibitors. In some instances, the YAP inhibitor is a small molecule agent that exhibits the desired activity, e.g., inhibiting YAP expression and/or activity. Naturally occurring or synthetic small molecule compounds of interest include numerous chemical classes, such as organic molecules, e.g., small organic compounds having a molecular weight of more than 50 and less than about 2,500 Daltons. Candidate agents have functional groups for structural interaction with proteins, particularly hydrogen bonding, and typically include at least an amine, carbonyl, hydroxyl or carboxyl group, preferably at least two of the functional chemical groups. The candidate agents may include cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Such molecules may be identified, among other ways, by employing the screening protocols.

In some cases, the YAP inhibitor is a photosensitizing agent. In some cases, the YAP inhibitor is a benzoporphyrin derivative (BPD). The benzoporphyrin derivative may be any convenient benzoporphyrin derivative such as, e.g., those described in U.S. Pat. Nos. 5,880,145; 6,878,253; 10,272,261; and U.S. Application No. 2009/0304803, the disclosures of which are incorporated herein by reference in their entireties. In some cases, the benzoporphyrin derivative is a photosensitizing agent. In some cases, the YAP inhibitor is verteporfin (benzoporphyrin derivative monoacid ring A, BPD-MA; tradename: Visudyne®).

Further details regarding YAP inhibitors and methods of using the same are provided in U.S. patent application Ser. No. 17/626,699; the disclosure of which is herein incorporated by reference.

In some instances, aspects of the methods may include administering an effective amount of a mechanotransduction blocker in combination with a Piezo inhibitor. In certain embodiments, the Piezo inhibitor includes a Piezo1 and/or Piezo2 inhibitor. In some cases, the Piezo inhibitor is a Piezo1 inhibitor. In some cases, the Piezo inhibitor is a Piezo2 inhibitor. In some case, both a Piezo1 inhibitor and Piezo2 inhibitor are administered to a subject. In some cases, the method consists essentially of administering a Piezo inhibitor. As used herein, a “Piezo inhibitor” refers to a molecule that may inhibit Piezo protein function and signaling. In some cases, the Piezo inhibitor inhibits cellular mechanical signaling. In some cases, the Piezo inhibitor reduces or inhibits Piezo protein expression (DNA or RNA expression) or activity (e.g., nuclear translocation). In some cases, the Piezo inhibitor reduces or inhibits the interaction of a Piezo protein with other signaling molecules. In certain embodiments, administering the Piezo inhibitor reduces mechanical activation of one or more cells, e.g., adipocytes, in a wound, wherein, e.g., the level of mechanical activation of the one or more cells, e.g., adipocytes, in a wound is reduced compared to a suitable control. Further details regarding Piezo inhibitors and methods of using the same are provided in U.S. Provisional Patent Application Ser. No. 63/335,843; the disclosure of which is herein incorporated by reference.

In the context of a combination therapy, combination therapy compounds may be administered by the same route of administration (e.g. intrapulmonary, oral, enteral, etc.) that the mechanotransduction blocker is administered. In the alternative, the compounds for use in combination therapy with the mechanotransduction blocker may be administered by a different route of administration.

### Pharmaceutical Compositions

As summarized above, pharmaceutical compositions are provided for practicing the methods disclosed herein. Pharmaceutical compositions comprise the mechanotransduction blocker of the present disclosure and a pharmaceutical acceptable excipient(s).

A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3^{rd} ed. Amer. Pharmaceutical Assoc.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

The pharmaceutical compositions of the present disclosure comprise a mechanotransduction blocker. Mechanotransduction blockers that find use in the present disclosure are any blockers that impair mechanotransduction signaling pathways. Non-limiting examples of mechanotranduction blockers include, without limitation, integrin inhibitors, focal adhesion kinase (FAK) inhibitors, Talin inhibitors, Vinculin inhibitors, Paxillin inhibitors, Zyxin inhibitors, VASP inhibitors, p130^{cas} inhibitors, etc. In some embodiments, the mechanotransduction inhibitor is a focal adhesion kinase (FAK) inhibitor. Non-limiting examples of FAK inhibitors include, without limitation, PF-56227, PF-573228, TAE226 (NVP-TAE226), BI-4464, GSK2256098, PF-431396, PND-1186 (VS-4718), Y15, Defactinib (VS-6063), Solanesol (Nonaisoprenol), etc. In addition to the FAK inhibitors disclosed above, other types of inhibitors may be used. For example, other types of FAK inhibitors include, without limitation, siRNA, anti-sense oligonucleotides (ASO). CRISPR-mediated knockout or knockdown of FAK, etc.

In some embodiments, the pharmaceutical composition includes a sustained release formulation. Sustained release formulations of the present disclosure are any sustained release formulation that is capable of releasing the mechanotransduction blocker for a prolonged period of time. The sustained release formulation is capable of releasing the mechanotransduction blocker for a range of time. For instance, the sustained release formulation may release the mechanotransduction inhibitor for at least 12 hours, at least 24 hours, at least 36 hours, at least 48 hours, at least 60 hours, at least 72 hours, at least 84 hours, at least 96 hours or greater than 96 hours.

The sustained release formulation of the present disclosure is capable of releasing the mechanotransduction blocker into a specified depth into the skin grafts. The depth into the skin graft is a measure of the distance from the stratum corneum to the farthest point into the tissue beneath the stratum corneum. In some embodiments, the specified depth that the sustained release formulation releases into the skin graft is at least 0.5 mm, at least 0.6 mm, at least 0.7 mm, at least 0.8 mm, at least 0.9 mm, at least 1 mm, at least 1.1 mm, at least 1.2 mm, at least 1.3 mm, at least 1.4 mm, at least 1.5 mm, at least 1.6 mm, at least 1.7 mm, at least 1.8 mm, at least 1.9 mm, at least 2 mm, at least 2.1 mm, at least 2.2 mm, at least 2.3 mm, at least 2.4 mm, at least 2.5 mm, at least 2.6 mm, at least 2.7 mm, at least 2.8 mm, at least 2.9 mm, at least 3 mm, or greater than 3 mm.

In some embodiments, the sustained release formulation contains a gel formulation. In some embodiments, the gel formulation includes a hydrogel, e.g., a carbohydrate based hydrogel, a protein based hydrogel, etc. In some embodiments, the hydrogel contains a biodegradable pullulan-based hydrogel. Pullulan-based hydrogels are known in the art and have been described in Wong et al. (Tissue Eng Part A. 2011 March; 17(5-6):631-44) and Wong et al. (Macromol Biosci. 2011 Nov. 10; 11(11):1458-66).

In some embodiments, the pharmaceutical composition is formulated in an aqueous buffer. Suitable aqueous buffers include, but are not limited to, acetate, succinate, citrate, and phosphate buffers varying in strengths from 5 mM to 100 mM. In some embodiments, the aqueous buffer includes reagents that provide for an isotonic solution. Such reagents include, but are not limited to, sodium chloride; and sugars e.g., mannitol, dextrose, sucrose, and the like. In some embodiments, the aqueous buffer further includes a non-ionic surfactant such as polysorbate 20 or 80. Optionally the pharmaceutical composition may further include a preservative. Suitable preservatives include, but are not limited to, a benzyl alcohol, phenol, chlorobutanol, benzalkonium chloride, and the like. In many cases, the formulation is stored at about 4° C. Pharmaceutical compositions may also be lyophilized, in which case they generally include cryoprotectants such as sucrose, trehalose, lactose, maltose, mannitol, and the like. Lyophilized formulations can be stored over extended periods of time, even at ambient temperatures.

In some embodiments, the mechanotransduction blocker is formulated with a second agent such as the combination therapies disclosed above in a pharmaceutically acceptable excipient(s).

The subject pharmaceutical composition can be administered orally, subcutaneously, intramuscularly, parenterally, or other route, including, but not limited to, for example, oral, rectal, nasal, topical (including transdermal, aerosol, buccal and sublingual), vaginal, parenteral (including subcutaneous, intramuscular, intravenous and intradermal), intravesical or injection into an affected organ.

Each of the active agents can be provided in a unit dose of from about 0.1 μg, 0.5 μg, 1 μg, 5 μg, 10 μg, 50 μg, 100 μg, 500 μg, 1 mg, 5 mg, 10 mg, 50, mg, 100 mg, 250 mg, 500 mg, 750 mg or more.

The pharmaceutical composition may be administered in a unit dosage form and may be prepared by any methods well known in the art. Such methods include combining the mechanotransduction blocker with a pharmaceutically acceptable carrier or diluent which constitutes one or more accessory ingredients. A pharmaceutically acceptable carrier is selected on the basis of the chosen route of administration and standard pharmaceutical practice. Each carrier must be “pharmaceutically acceptable” in the sense of being compatible with the other ingredients of the formulation and not injurious to the subject. This carrier can be a solid or liquid and the type is generally chosen based on the type of administration being used.

Examples of suitable solid carriers include lactose, sucrose, gelatin, agar and bulk powders. Examples of suitable liquid carriers include water, pharmaceutically acceptable fats and oils, alcohols or other organic solvents, including esters, emulsions, syrups or elixirs, suspensions, solutions and/or suspensions, and solution and or suspensions reconstituted from non-effervescent granules and effervescent preparations reconstituted from effervescent granules. Such liquid carriers may contain, for example, suitable solvents, preservatives, emulsifying agents, suspending agents, diluents, sweeteners, thickeners, and melting agents. Preferred carriers are edible oils, for example, corn or canola oils. Polyethylene glycols, e.g. PEG, are also good carriers.

Any drug delivery device or system that provides for the dosing regimen of the instant disclosure can be used. A wide variety of delivery devices and systems are known to those skilled in the art.

### Kits

Also kits for practicing the methods described in the present disclosure. In general, subject kits may include the pharmaceutical composition described above as described above and a skin graft harvester. The pharmaceutical composition may be contained in a specific deliver device. Delivery devices include, without limitation, patches, gauze dressings, transparent film dressings, foam dressings, hydrocolloids dressings, alginate dressings, composite dressings, etc.

The skin graft harvester of the present disclosure is any skin graft harvester capable of producing a split-thickness skin graft. Skin graft harvesters that find use in the present disclosure include, without limitation, a surgical knife, oscillating, Goulian knife, an air powered dermatome, an electric powered dermatome, etc.

A subject kit can include any combination of components for performing the methods of the present disclosure. The components of a subject kit can be present as a mixture or can be separate entities. In some cases, components are present as a lyophilized mixture. In some cases, the components are present as a liquid mixture. In some cases, the components are present as a semi-sold mixture such as a hydrogel. Components of a subject kit can be in the same or separate containers, in any combination.

The subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a remote site.

The following example(s) is/are offered by way of illustration and not by way of limitation.

### Examples

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference. Reagents, cloning vectors, cells, and kits for methods referred to in, or related to, this disclosure are available from commercial vendors such as BioRad, Agilent Technologies, Thermo Fisher Scientific, Sigma-Aldrich, New England Biolabs (NEB), Takara Bio USA, Inc., and the like, as well as repositories such as e.g., Addgene, Inc., American Type Culture Collection (ATCC), and the like.

### I. Disrupting Mechanotransduction Decreases Fibrosis and Contracture in Split Thickness Skin Grafting

### A. Abstract

Burns and other traumatic injuries represent a substantial biomedical burden. The current standard-of-care for deep injuries is autologous split-thickness skin grafting (STSG), which frequently results in contractures, abnormal pigmentation, and loss of biomechanical function. Currently, there are no effective therapies that can prevent fibrosis and contracture after STSG. Here, we have developed a clinically relevant porcine model of STSG and comprehensively characterized porcine cell populations involved in healing with single cell resolution. We identified an upregulation of pro-inflammatory and mechanotransduction signaling pathways in standard split thickness skin grafts. By blocking mechanotransduction using a small molecule focal adhesion kinase (FAK) inhibitor, we promoted healing, reduced contracture, mitigated scar formation, restored collagen architecture, and ultimately improved graft biomechanical properties. Acute mechanotransduction blockade upregulated myeloid CXCL10-mediated anti-inflammation with decreased CXCL14-mediated myeloid and fibroblast recruitment. At later time points, mechanical signaling shifted fibroblasts toward pro-fibrotic differentiation fates, whereas disruption of mechanotransduction modulated mesenchymal fibroblast differentiation states to block those responses and instead drove fibroblasts toward pro-regenerative, adipogenic states similar to unwounded skin. We then confirmed these two diverging fibroblast transcriptional trajectories in human skin, human scar, and a three dimensional organotypic model of human skin. Taken together, pharmacological blockade of mechanotransduction markedly improved large animal healing after STSG by promoting both early, anti-inflammatory and late, regenerative transcriptional programs, resulting in healed tissue similar to unwounded skin. FAK inhibition is a supplement the current standard of care for traumatic and burn injuries.

### B. Materials and Methods

### 1. Study Design

The overall goal of the study was to identify and therapeutically target molecular drivers of fibrosis in large organisms to improve healing outcomes after STSG. Each pig (n=5) received six STSG (n=30 STSG biological replicates total). Treatment conditions were randomly assigned to different STSG across each pig dorsum. All assessments of various scar properties (wound contracture, VAS, re-epithelialization, and engraftment) were performed using images blinded to the observer. Analysis of collagen architecture was performed in an unbiased manner using quantitative computer algorithms CurveAlign and CT-FIRE. 10× genomics scRNA-seq data was captured and sequenced by the Stanford Functional Genomics Facility, who were blinded to the treatment groups. scRNA-seq data were analyzed using Seurat, a quantitative analysis package for clustering and embedding scRNA-seq data. For in vitro experiments, (n=15) human collagen hydrogel biological replicates, (n=6) porcine hydrogel biological replicates were created, and each hydrogel was randomly assigned to the different treatment groups. No data were excluded. These sample sizes were large enough to detect the effect of treatment across a range of variables. All animal work was performed in accordance with Stanford APLAC and AAALAC guidelines (APLAC protocols 31530 and 32962). Human tissue samples were collected under IRB #54225 from procedures in which the samples would otherwise be discarded. Patient identifying information was not recorded for any of the samples.

### 2. Development of a Translational Porcine Model for STSG

We developed a novel porcine STSG model using surgical techniques commonly applied for the clinical treatment of burn wounds and other soft-tissue defects. First, we created full-thickness excisional wounds measuring 25 cm² on the backs of adult red Duroc pigs, leaving the underlying muscle fascia intact. In the same surgery, we harvested thin STSG (0.01 in) from the same pig using a clinical-grade electric dermatome (Zimmer Biomet). First, the unwounded skin of the donor site was lubricated (Surgilube) and the dermatome was passed over the skin at a controlled rate. The resultant skin was placed on a Skin Graft Carrier (Dermacarrier II, Zimmer 00770800010) and slowly fed through a Skin Graft Mesher (Zimmer 7701) to create a mesh graft of 1:1.5 ratio (41). The grafts were carefully spread out on the graft carrier and placed onto the exposed muscle fascia of the wound beds. Skin staples (Covidien 8886803712) were used to secure the graft to the wound bed (about 5 staples per edge). The grafts were covered with three layers of petrolatum gauze (Xeroform, Covidien SH84-433605) to prevent them from drying out and also to prevent bacterial infection. Bolster dressings were prepared by cutting 5.5 cm×5.5 cm squares from VAC Granufoam (Small Dressing Kit, Acelity M8275065) and heavily secured on top of the gauze and STSG with additional skin staples. Finally, Telfa non-adherent dressings (Covidien 1961) and Tegaderm adherent dressings (3M 1624W) were used to cover the bolster sponge dressings. In order to prevent irritation and to minimize the animal's ability to impact the dressings, custom-designed polyester jackets tailored to the individual pigs were utilized (Lomir Biomedical Inc). Animals were given oral amoxicillin 10 mg/kg post-operatively twice a day for 5 days total.

For treatment experiments, (n=5) pigs were used, and each pig received 6 total STSG. For each pig for POD90 experiments, STSG were randomly assigned to equally receive either FAKI hydrogels, blank hydrogels, or no hydrogel (standard dressings used for all STSG), with treatment conditions randomly assigned to different STSG across each pig dorsum. For each pig for POD7 and POD14 experiments, STSG were randomly assigned to equally receive either FAKI hydrogels or no hydrogel. For all experiments, treatment conditions were randomly assigned to different STSG across each pig dorsum to minimize any locational effects. Hydrogels were applied over the STSG before the standard dressing (petrolatum gauze+bolster sponge+Telfa+Tegaderm+custom jacket). Sterile hydrogels were pre-soaked in sterile saline before application. STSG were not disturbed during the first 3 days. Then, dressings and hydrogels were changed every other day for the first three weeks after initial injury until week 3. Each of these dressing changes were performed under sterile conditions. Skin staples were removed with a skin stapler remover (3M MMMSR3Z), the wound and graft were gently irrigated with sterile saline. Photos were taken at each dressing change, and hydrogels and bolster dressings were replaced with fresh dressings. Animals were subject to short-term sedation for each dressing change.

After three weeks, dressings (and hydrogels) were changed twice per week until month 3. STSG were no longer bolster dressed and instead a conventional dressing was used (Telfa+Tegaderm). Each STSG within the animal was biopsied for histological and molecular evaluation at the end of the study.

### 3. Statistics

Statistical analysis was performed in Prism8 (GraphPad, San Diego, California). When comparing two samples, a t test was used. When comparing more than two samples, either a one- or two-way analysis of variance (ANOVA) was used, with Tukey's multiple comparisons test. Data are presented as means±SEM. P values of P<. 05 were considered statistically significant.

### C. Results

### 1. Porcine Model of Autologous Split Thickness Skin Grafting

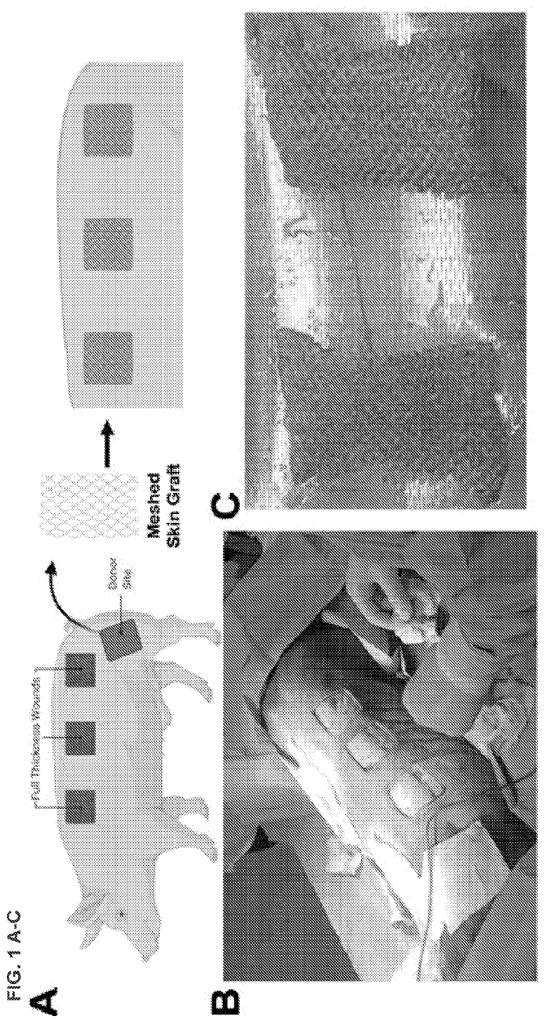

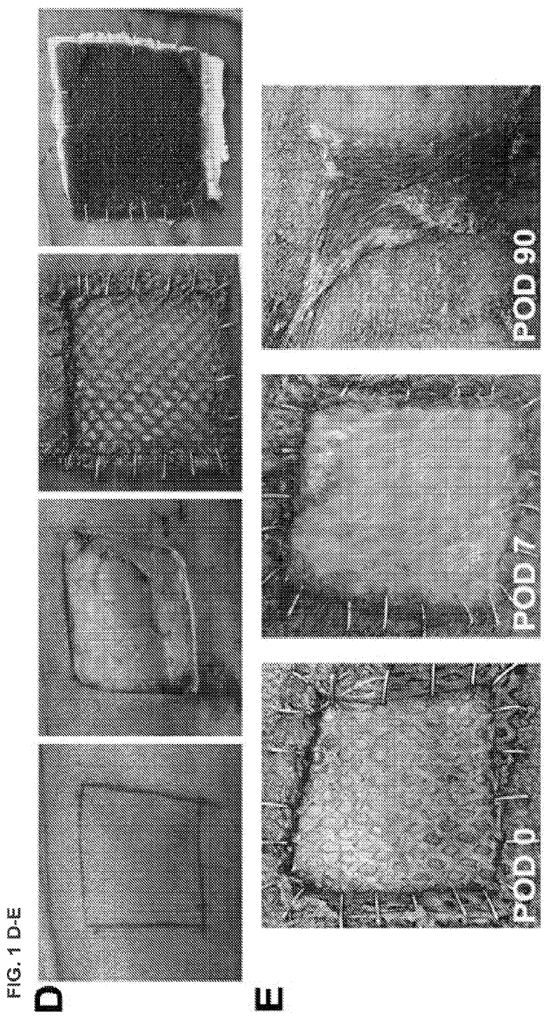

We developed a clinically relevant porcine STSG model using standardized surgical techniques applied for the clinical treatment of burn wounds and other soft-tissue defects. We elected to use the red Duroc pig due to its close recapitulation of human skin physiology, cutaneous wound healing kinetics, and scar formation after injury (29, 37) (FIG. 1). Human and red Duroc skin have similar thickness and biomechanical properties, and, unlike small animal models, both heal from deep dermal injuries by developing thick, fibrotic scar tissue that stiffens and contracts over time (38).

To treat full thickness injuries such as severe burns, surgeons sometimes excise the necrotic, injured tissue to expose the underlying muscle fascia before applying the STSG (39, 40). To mimic this, we created full-thickness excisional wounds measuring 25 cm² on the backs of adult red Duroc pigs (FIGS. 1, A and B), leaving the underlying muscle fascia intact. In the same surgery, we harvested STSGs (0.01 in) using a clinical-grade electric dermatome (Zimmer Biomet) and meshed the resulting grafts at a 1:1.5 ratio (41) (FIG. 1C). Clinically, STSGs (˜0.01 in) are usually harvested to contain the epidermis and superficial dermis, allowing the donor site to re-epithelialize and heal (41, 42). These STSGs were then applied directly to the dorsal wounds, using petrolatum gauze, a bolster dressing, and skin staples to maximize engraftment (FIG. 1D). Each animal also wore a custom-made, compressive jacket throughout the study. All STSGs experienced complete engraftment (incorporation). We initially observed the typical “meshed” appearance of the healing grafts, characteristic of human STSG during the early healing phase, followed by substantial graft contracture and scar contracture with hyperpigmentation and a rough texture (FIG. 1E). These observations coincided with the time course of skin healing and contracture after STSG in humans (43).

2. Custom Single Cell RNA Sequencing Methods for Porcine Skin Tissue Identify Cellular Subpopulations that Contribute to Scarring

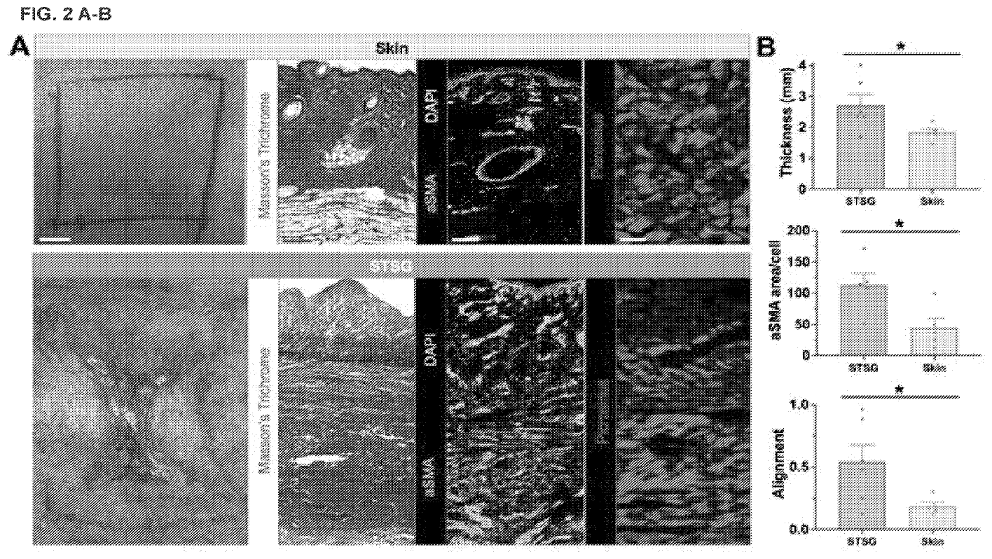

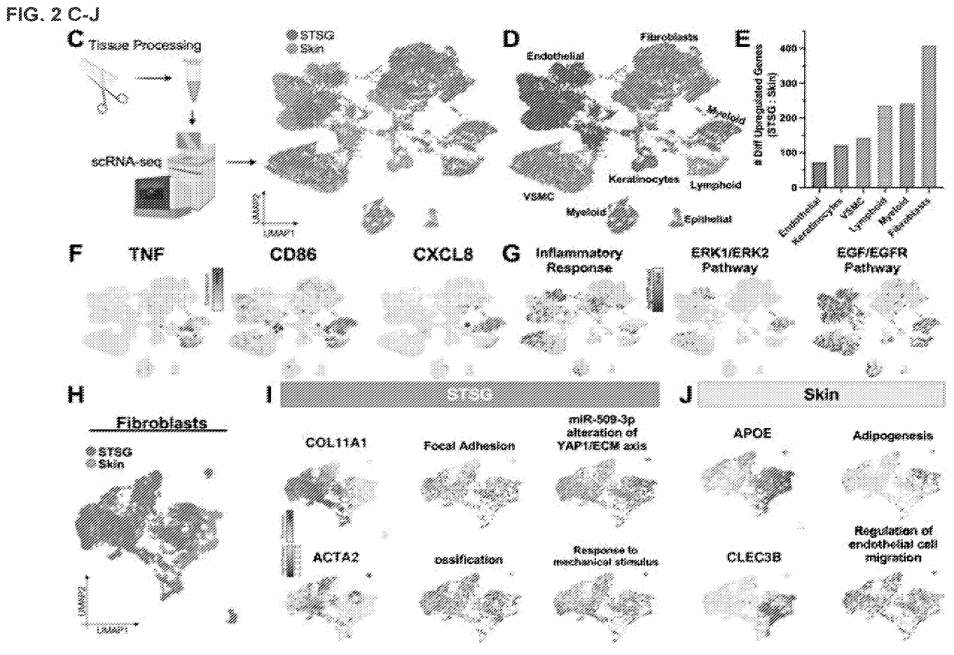

STSGs did not restore normal skin appearance and instead exhibited permanent scar contracture with excessive fibrosis, hyperpigmentation, and raised hypertrophic scar (HTS) formation at postoperative day (POD) 90 (FIG. 2A). Specifically, STSGs had a thickened dermis with more highly aligned collagen fibers and fewer dermal appendages, which are all classic signs of fibrotic healing. In contrast, normal unwounded skin had a randomly aligned, “basket-weave” collagen architecture (*P<0.05) (FIGS. 2, A and B). The STSG was also found to contain more pro-fibrotic myofibroblasts (*P<0.05) (FIGS. 2, A and B).

To further investigate the cellular and molecular changes that drove the differences between fibrotic STSG and unwounded skin, we performed scRNA-seq using the 10× Genomics platform following complete healing (FIG. 2C) (44, 45). Although previous studies have performed scRNA-seq on unwounded human skin (46-48) or compared human keloid to human scar (49), none have directly compared fibrotic tissue against unwounded skin with single cell resolution before. To collect high-quality cells, we optimized our previously published methods and harvested porcine tissue from both late stage (POD 90) STSG scar and normal unwounded skin (44).

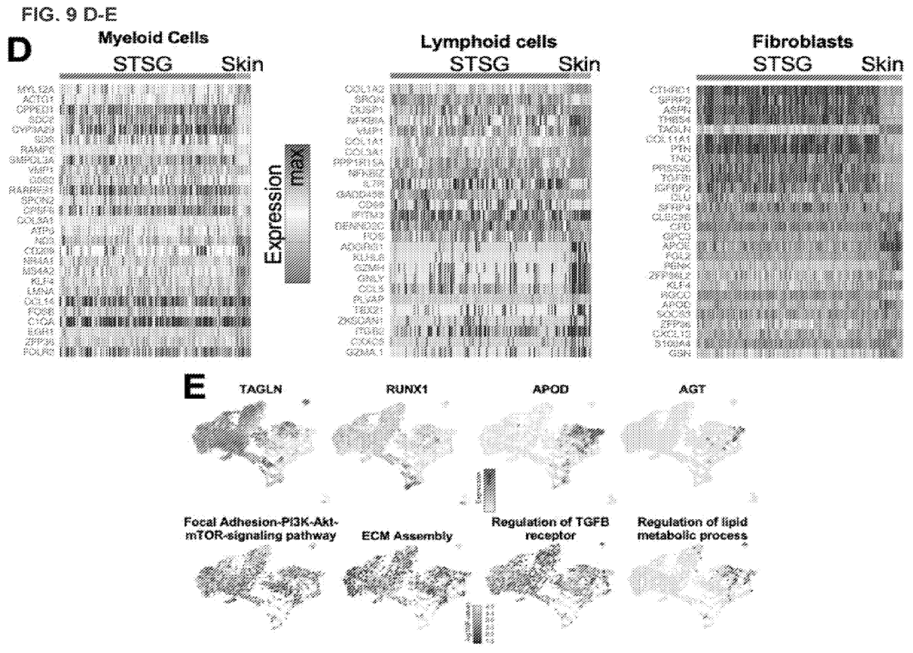

We created a custom pig transcriptome generated using the Sus scrofa genome (Ensembl, v11.1) (50) in accordance with 10× protocols for non-model organisms (44), and we have made it publicly available (see Data and Materials Availability statement) with instructions to implement as part of a modified 10× CellRanger pipeline. Briefly, all read fragments were aligned to this transcriptome using standard base-matching thresholds (51), followed by single cell demultiplexing and unique molecular identifier (UMI) batch correction (52), allowing us to generate single cell mRNA count matrices for each pig sample. Data for individual cells from both groups were subjected to blinded Louvain-based clustering and embedded into a two-dimensional UMAP (uniform manifold approximation and projection) space (45) (FIG. 2C). A total of 7,700 cells were captured, and distinct populations of fibroblasts, myeloid cells, lymphoid cells, endothelial cells, vascular smooth muscle cells (VSMCs), and keratinocytes were identified using automated cell type annotations through the SingleR package (FIG. 2D) and verified with cell type-specific marker genes (FIG. 9A).

We observed that fibroblasts had over 400 differentially expressed genes (DEGs; average log fold change >0.5 between STSG and skin), and myeloid and lymphoid cells had over 200 DEGs between STSG and skin (FIG. 2E; FIG. 9B). In contrast, other cell types (keratinocytes, endothelial cells, VSMCs) had 100 or fewer DEGs, indicating that they were not transcriptionally different between normal skin and skin grafts (FIG. 2E; FIGS. 9, C and D). These findings suggested sustained elevation of fibroproliferative and inflammatory cell activity in the STSG at this late time point and corroborated our observation of increased numbers of myofibroblasts in STSG compared to unwounded skin (FIGS. 2, A and B).

3. Fibrosis Following STSG is Associated with Increased Mechanotransduction Signaling

We first examined the myeloid cells in our STSG and unwounded skin scRNA-seq datasets. We used Genetrail3, a pipeline for over-representation analysis (ORA) to explore differential regulation of signaling pathways (53). First, we observed that STSG myeloid cells demonstrated an inflammatory phenotype characterized by upregulation of inflammatory markers such as CXCL8 and CD86 as well as enrichment for immune response-activating signal transduction (GO-BP:0002758) and inflammatory response pathways (WP453) (54) (FIGS. 2, F and G). Myeloid STSG cells were also enriched for fibrosis-driving genes, such as TNF (55), and demonstrated an upregulation of common mechanotransduction pathways such as the ERK1/ERK2 and EGF/EGFR pathways (56) (FIGS. 2, F and G). These pathways are downstream of FAK and suggest that these immune cells are mechanoresponsive even at later time points (57, 58).

Next, we compared the transcriptional profiles of fibroblasts in STSG and unwounded skin following complete healing over 90 days (FIG. 2H). These revealed a marked elevation in the expression of myofibroblast-associated and collagen-producing genes such as ACTA2 (encoding smooth muscle alpha actin, aSMA), RUNX1 (runt-related transcription factor 1), TAGLN (transgelin), and COL11A1 (collagen type XI alpha 1 chain), suggesting that fibroblasts in STSGs had differentiated into more contractile myofibroblast phenotypes associated with increased collagen production and fibrosis, which was also supported by our immunofluorescent staining (59) (FIGS. 2, A, B, and I; FIG. 9E). STSG fibroblasts exhibited an enrichment of gene sets related to mechanotransduction and collagen production/organization driving scar formation, such as response to mechanical stimulus (GO-BP:0009612), focal adhesion (WP306), ECM assembly (GO-BP:0030198), YAP signaling (WP3967), response to TGFB signaling (WP560), and ossification (GO-BP:0001503) (FIG. 2I). STSG fibroblasts were also characterized by a downregulation of genes associated with adipogenesis (WP236), lipid transport (GO-BP:0006869), and regulation of endothelial cell migration (GO-BP:0010594), defined by regenerative, adipogenic markers such as APOE (apolipoprotein E), APOD (apolipoprotein D), CLEC3B (c-type lectin domain family 3 member B), and AGT (angiotensinogen) (60) (FIG. 2J; FIG. 9E). These pathways and genes suggested that STSG fibroblasts were shifted away from the more homeostatic, quiescent baseline observed in normal skin.

Although fibroblasts are generally regarded as the primary mediators of collagen deposition and scar contracture (26), recent studies have suggested a role for immune cells in regulating (and sustaining) fibrotic processes (61-64). Although there is some evidence that macrophages respond to mechanical cues in certain situations, these studies have yielded conflicting results, with literature suggesting that mechanical strain produces both pro- and anti-inflammatory responses (57, 58). Overall, our transcriptomic data indicated that tissue that develops after STSG is different from unwounded skin and primarily characterized by increased mechanotransduction signaling in both inflammatory cells and fibroblasts, suggesting a shared common pathway in the context of STSG. Although the importance of mechanotransduction has previously been identified during the healing of open wounds (23, 25-27), the contributions of mechanotransduction signaling after skin grafting have yet to be investigated. To develop therapeutics that improve STSG outcomes, we hypothesized that targeting mechanotransduction signaling might improve healing and reduce scar formation after STSG.

### 4. Disrupting Mechanotransduction Mitigates Fibrosis in STSG by Reducing Contracture, Promoting Engraftment, and Improving Biomechanical Properties

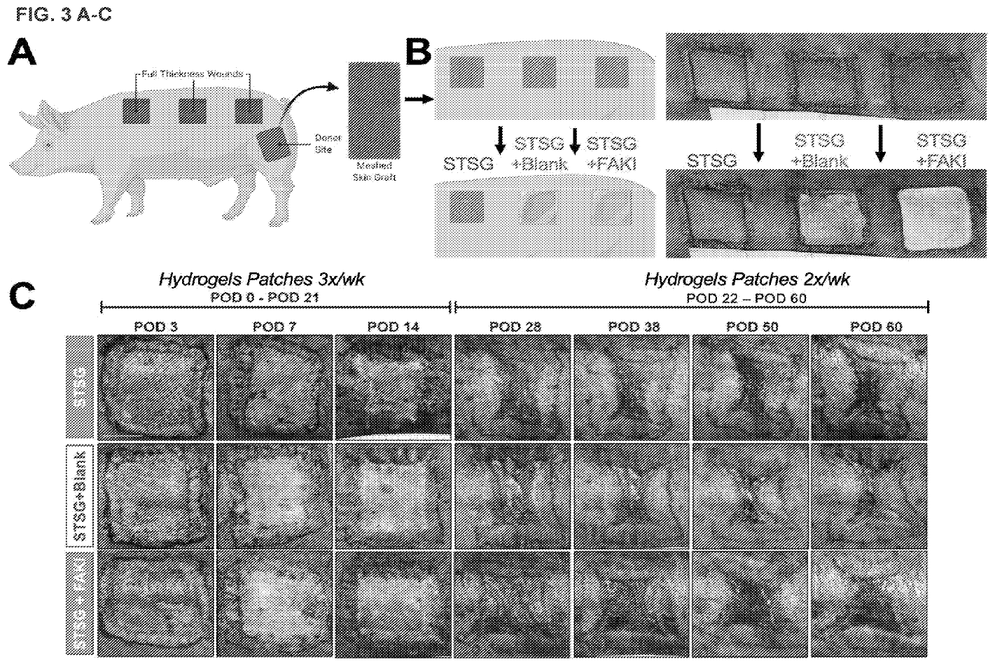

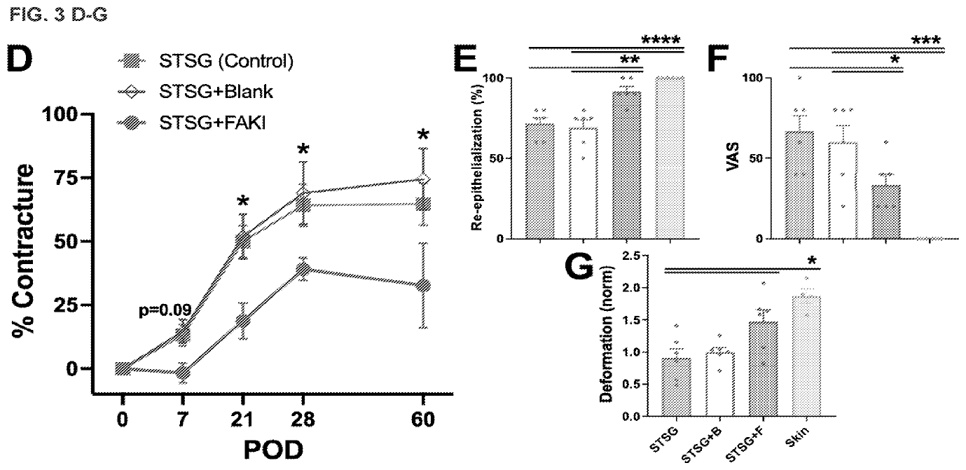

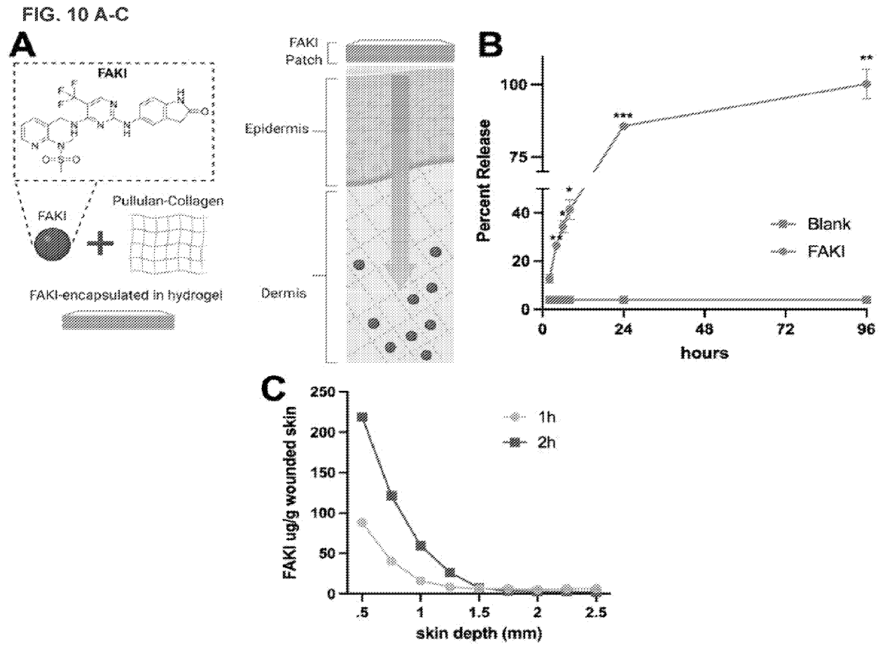

To inhibit mechanotransduction in porcine wounds treated with STSG, we delivered FAKI (VS-6062) using a biodegradable, biocompatible, and soft pullulan-based hydrogel optimized for sustained drug release during wound healing (23) (FIGS. 3, A and B). FAK is a critical transducer of integrin-matrix forces to downstream intracellular pathways (26). VS-6062 (formerly Pfizer PF-00562271) is a potent, ATP-competitive, later generation small molecule FAKI that blocks tumor growth and has undergone Phase I trials against advanced solid tumors (ClinicalTrials.gov Identifier: NCT00666926) (65). VS-6062 also has a strong selectivity for FAK relative to a wide range of other kinase targets (66, 67), and we have previously characterized the release of this drug in our hydrogel (23, 26). The hydrogel contains VS-6062 and is rehydrated in saline to convert it into a hydrogel dressing (FIG. 10A) that slowly releases the drug, which permeates through the STSG dermis over time (FIGS. S2, B and C). Similar to other hydrogels, this hydrogel dressing provides coverage over a wound or STSG, preventing desiccation and facilitating moist wound healing (FIG. 3B).

We measured the surface area of the scars and found that FAK inhibition blocked scar contracture at early time points (POD7, P=0.09; POD21, *P<0.05) (FIGS. 3, C and D). In contrast, STSGs and STSGs treated with blank hydrogels had immediate scar contraction by POD7, which continued to increase over time. At POD28, untreated and blank hydrogel-treated STSGs contracted over 70%, whereas the FAKI-treated STSGs exhibited only 30% contracture (FIG. 3D). These observations were statistically significant and continued throughout all time points (*P<0.05).

A panel of three blinded plastic surgeons quantified STSG re-epithelialization and scar appearances using a Visual Analog Scale (VAS), a scar scoring system commonly used by physicians to stratify scar severity and visually assess scar appearance (range of 0-100, with 0=unwounded skin and 100=hypertrophic scar) (68). From these blinded scores, we found that FAKI hydrogels accelerated interstitial re-epithelialization of STSGs at POD 7 (**P<0.01) (FIG. 3E). FAKI hydrogels also significantly improved the fibrotic appearance of STSG wounds over time, demonstrating decreased scar formation (*P<0.05) (FIG. 3F).

Using a tissue cutometer (non-invasive clinical instrument that measures the viscoelastic properties of skin through deformation with negative pressure), we found that FAKI-treated STSG were also less firm and less stiff than untreated STSG (*P<0.05) (FIG. 3G), exhibiting similar biomechanical properties to unwounded skin. Taken together, these data demonstrated that early intervention to disrupt cellular mechanotransduction pathways mitigated an array of complications that typically occur following STSG, including contracture, stiffness, and scar appearance.

5. Pharmacological Blockade of Mechanotransduction after STSG Restores Collagen Architecture Similar to Unwounded Skin

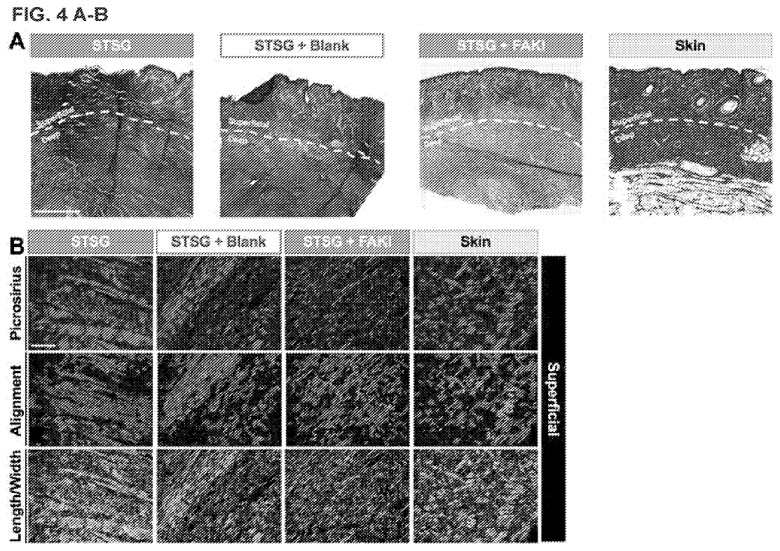

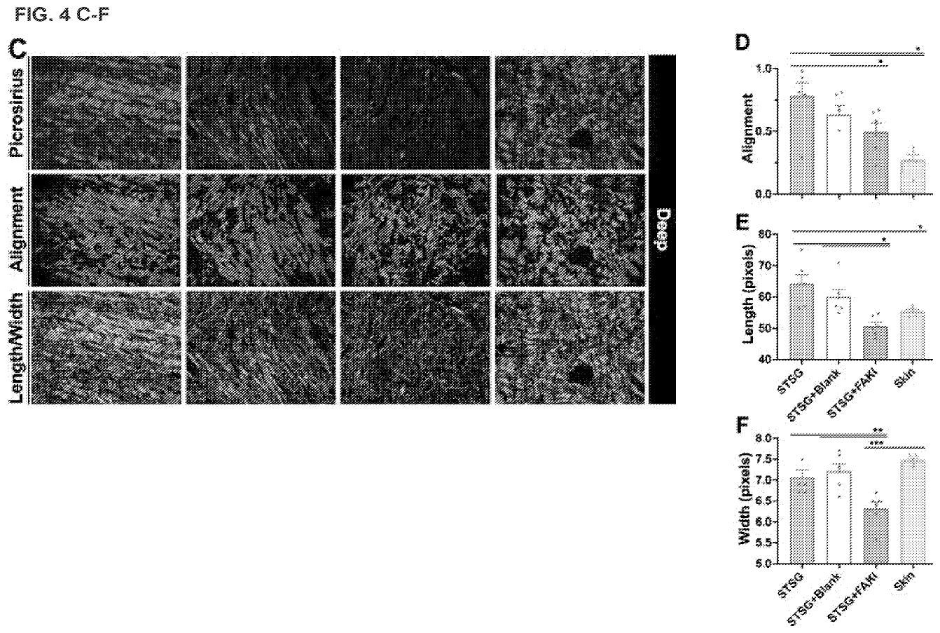

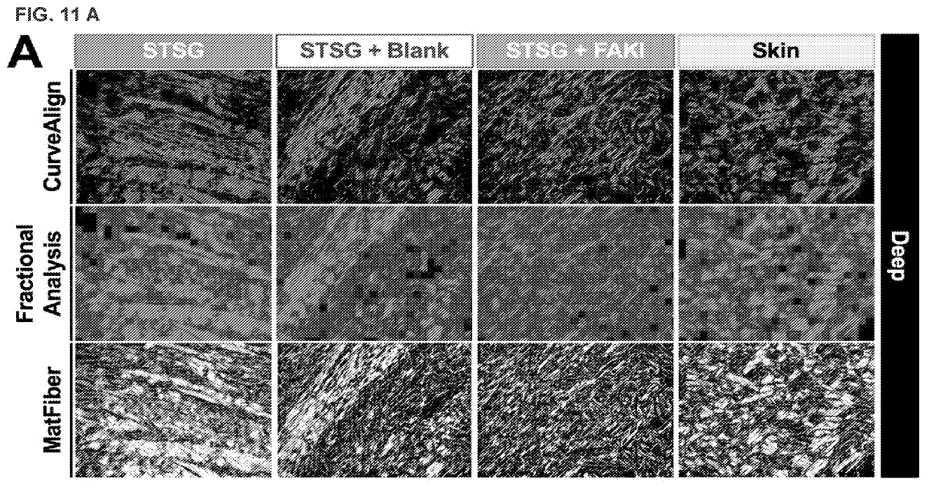

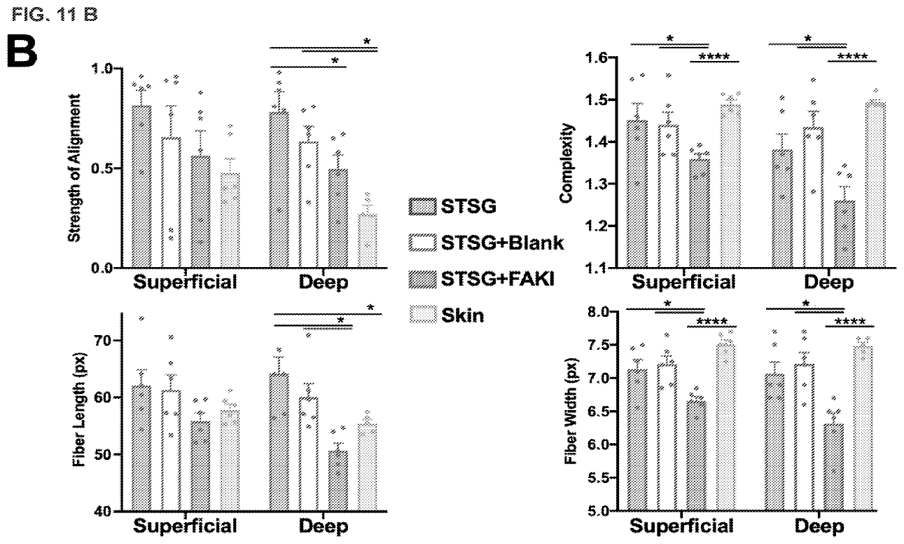

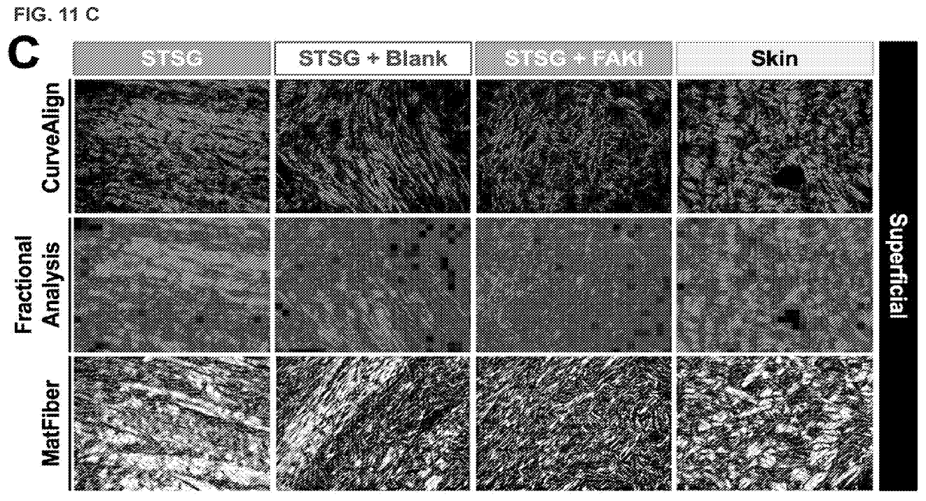

To confirm these gross anatomical findings, we used histological analysis to observe changes in tissue architecture after FAK inhibition (FIG. 4A). Pharmacological blockade of mechanotransduction significantly promoted shorter and more randomly aligned collagen in the deep dermis, similar to the typical basket weave-like collagen fiber network in unwounded skin (*P<0.05) (FIG. 4, B to E; FIGS. 11, A and B). Furthermore, FAK inhibition decreased overall collagen deposition throughout both the superficial and deep dermis by decreasing fiber widths and overall architectural complexity (**p<0.01, ***p<0.001) (FIG. 4F; FIGS. 11, B and C). Overall, FAKI-treated STSGs demonstrated dermal remodeling similar to that of unwounded skin throughout the entire thickness of the developing scar.

### 6. Mechanotransduction Blockade Causes an Acute Upregulation of Anti-Inflammatory Pathways in Myeloid Cells

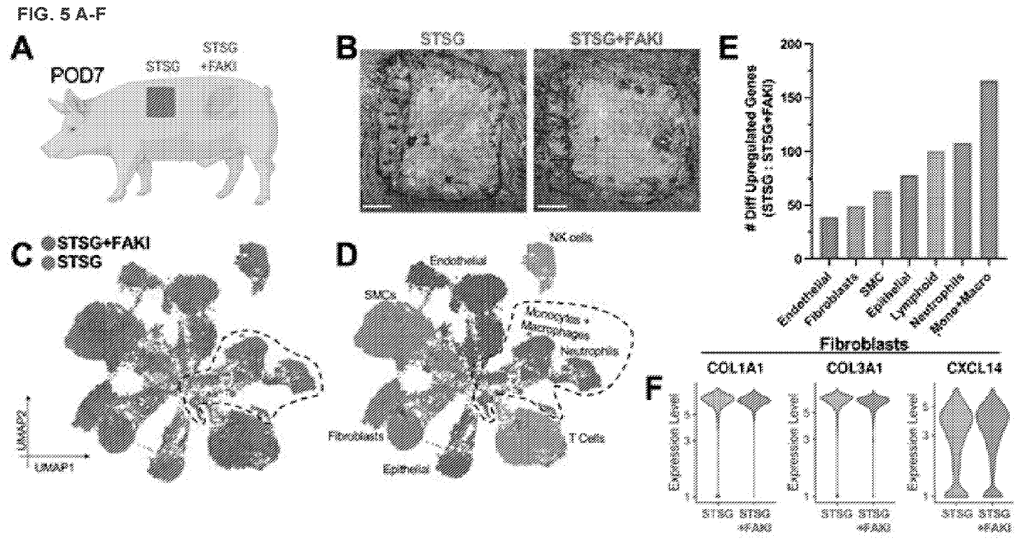

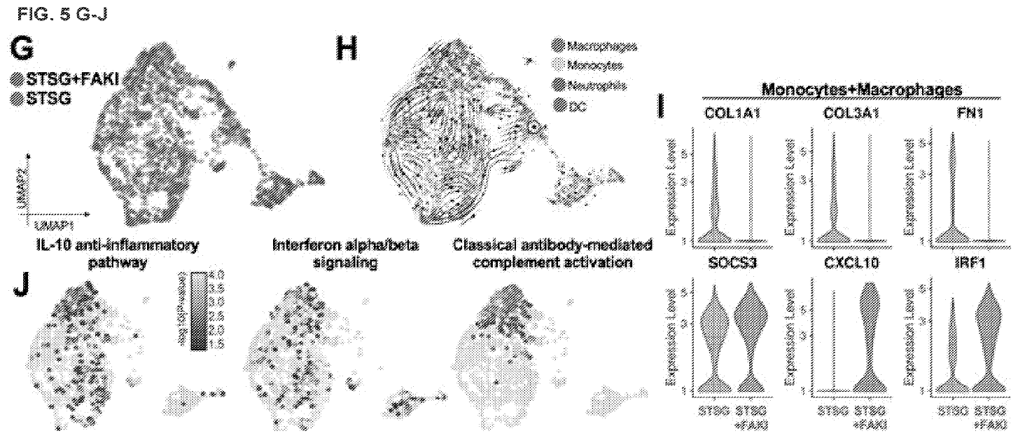

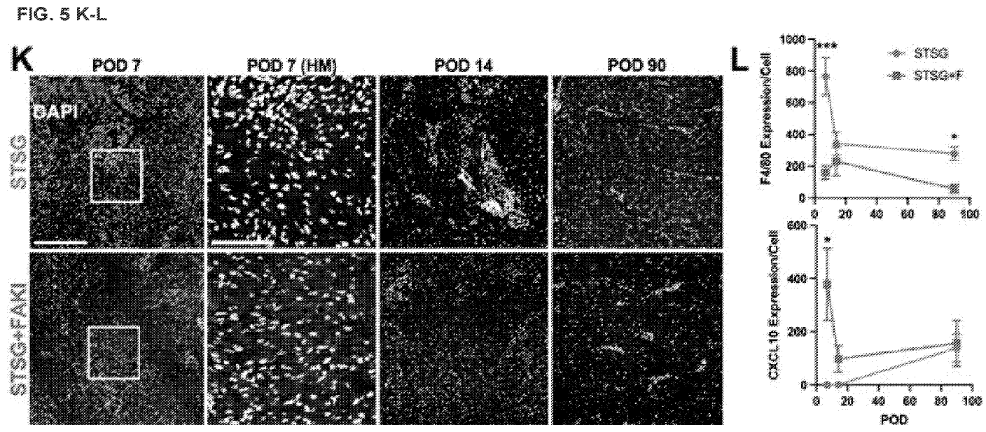

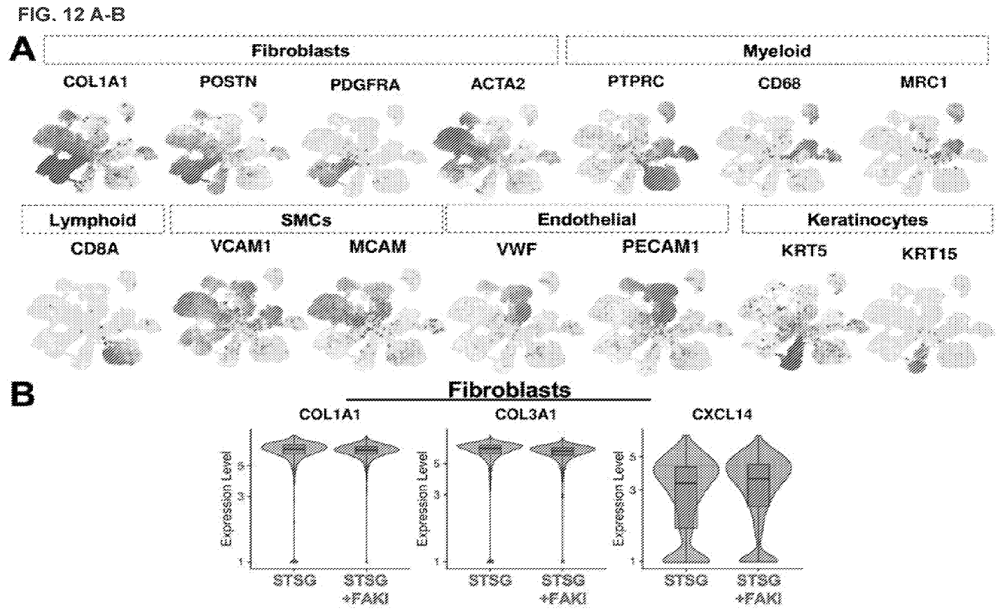

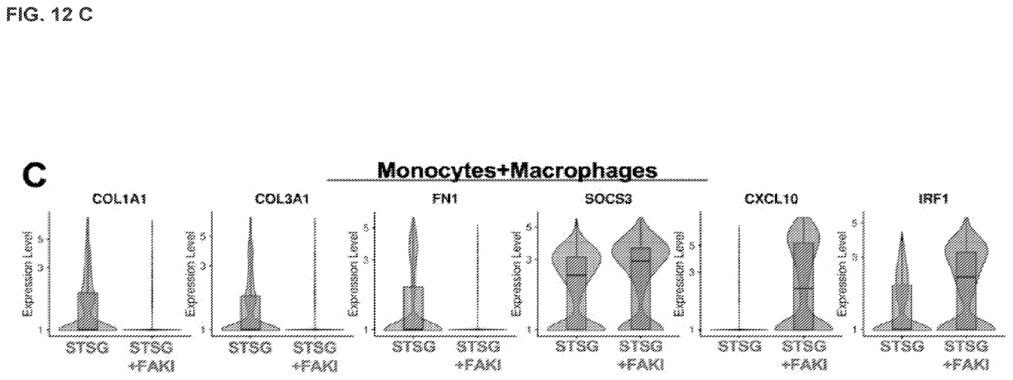

To understand the mechanisms driving these macro- and microscopic tissue changes, we examined how disrupting mechanotransduction affected healing within the cells (FIG. 5A). Since untreated (control) STSGs and STSGs treated with blank hydrogels demonstrated no differences across a wide range of clinical scar measurements (FIGS. 3 and 4), we focused our scRNA-seq analysis on cells from untreated STSG and STSG treated with FAKI. We first investigated the early stages of STSG incorporation in porcine tissue 7 days after STSG (FIGS. 5, A and B). Again, we captured a diverse cellular milieu in both treated and untreated STSG (FIGS. 5, C and D; FIG. 12), but the early timepoint monocyte-lineage cells (macrophages, monocytes, dendritic cells) had the most DEGs (>170) (FIG. 5E), followed by neutrophils and lymphoid cells (around 100 each), during this early inflammatory phase of wound healing (all other cell types <75). Unexpectedly, fibroblasts only had about 50 DEGs, indicating that there were not strong differences in fibroblast gene expression at these early time points, despite the substantial differences observed later. For example, in both conditions, fibroblasts exhibited similar expression of extracellular matrix markers such as COL1A1, COL3A1, FN1 (encodes for fibronectin), as well as inflammatory chemokines such as CXCL14 (69, 70) (FIG. 5F). These findings suggested that fibroblasts are not dramatically altered by mechanical signaling during the early stages of healing in this model.

Instead, in myeloid cells (monocyte-lineage and neutrophils), inhibiting mechanotransduction triggered a variety of beneficial transcriptional shifts (FIGS. 5, G and H). For example, STSG monocyte-lineage cells demonstrated an increased expression of COL1A1, COL3A1, and FN1 that was abrogated with FAK inhibition (FIG. 51), suggesting that FAK inhibition decreased early ECM deposition from myeloid cells. These findings suggest a previously underexplored ability of myeloid cells to contribute to extracellular matrix formation.

Disruption of mechanotransduction also upregulated myeloid CXCL10 expression (encodes for interferon gamma induced protein 10; IP-10) (FIG. 51), a secreted chemokine that inhibits fibroblast migration in response to pro-inflammatory markers (71). IP-10 therapy has previously been clinically used to reduce fibrosis (72-74). FAK inhibition also induced SOCS3 expression (FIG. 51), which is known to attenuate inflammatory IL6 expression (75). Using Genetrail3 to explore differential signaling pathways (53), we observed that FAK inhibited myeloid cells exhibited enrichment of gene sets related to the IL-10 Anti-inflammatory pathway (WP4495), Interferon alpha/beta signaling (WP1835), and Classical antibody-mediated complement activation (GO-BP:0006958) (FIG. 5J).

We then collected porcine STSG tissue across time (PODs 7, 14, 90) and mapped a time course of STSG healing at the protein level. Using immunofluorescent staining, we first investigated the presence of F4/80-positive macrophage populations within the tissue (FIG. 5K). Untreated STSG contained a significantly increased number of macrophages at early (POD 7, ***P<0.001) and late (POD 90, *P<0.05) time points, indicative of a chronic proliferative inflammatory response and supporting our earlier myeloid cell findings (FIG. 2, C to G). STSGs treated with FAKI significantly decreased the number of infiltrating inflammatory cells at both early (***P<0.001) and late (*P<0.05) time points (FIGS. 5, K and L). Of the inflammatory cells that remained, disruption of mechanotransduction significantly (*P<0.05) upregulated the secretion of IP-10 (CXCL10) at POD 7 (FIGS. 5, K and L), indicating decreased fibroblast recruitment into the wound over time and a reduction of downstream fibrosis.

Myeloid cells, which are involved in acute inflammation after soft tissue injury, have recently been identified as being mechanically sensitive in the context of a number of physiological processes in the body including proprioception, touch, balance, and hearing (3, 76, 77). Within the context of wound healing, recent studies have hinted that mechanotransduction may affect myeloid transcriptional dynamics and alter healing potential (57, 58, 78), but the mechanisms remain incompletely understood (79). Here, we observed that disruption of mechanotransduction had a greater effect on myeloid cells than fibroblasts at early time points by reducing inflammatory recruitment and promoting CXCL10-mediated anti-inflammatory transcriptional profiles. ECM-producing myeloid cells were recruited in excessive quantities to the STSG, indicating a previously unappreciated importance of myeloid cell collagen production during skin graft incorporation. These findings demonstrated that mechanically sensitive myeloid cells also respond to mechanotransduction blockade by promoting pro-regenerative phenotypes.

### 7. Disruption of Mechanotransduction Shifts Myofibroblast Transcriptional States Towards Regenerative Differentiation

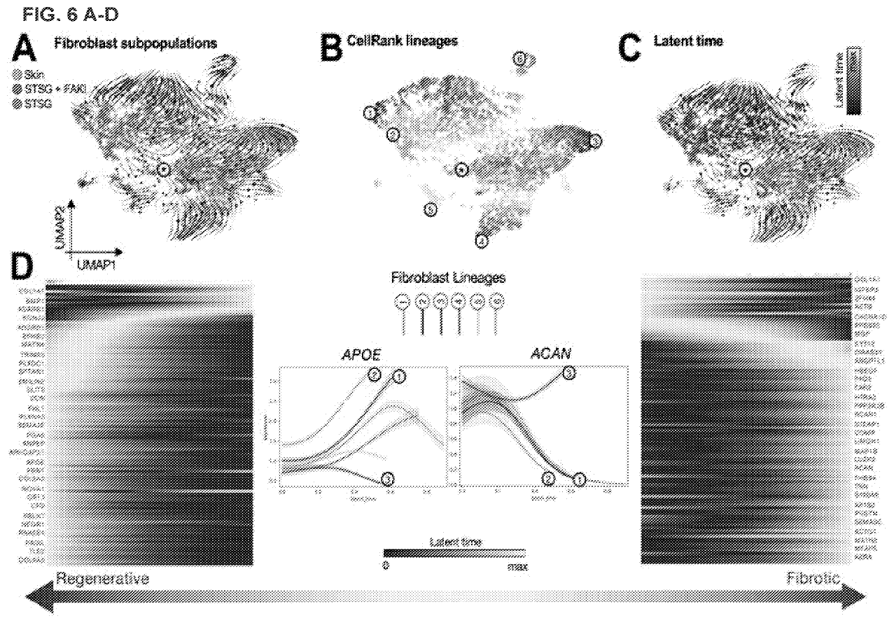

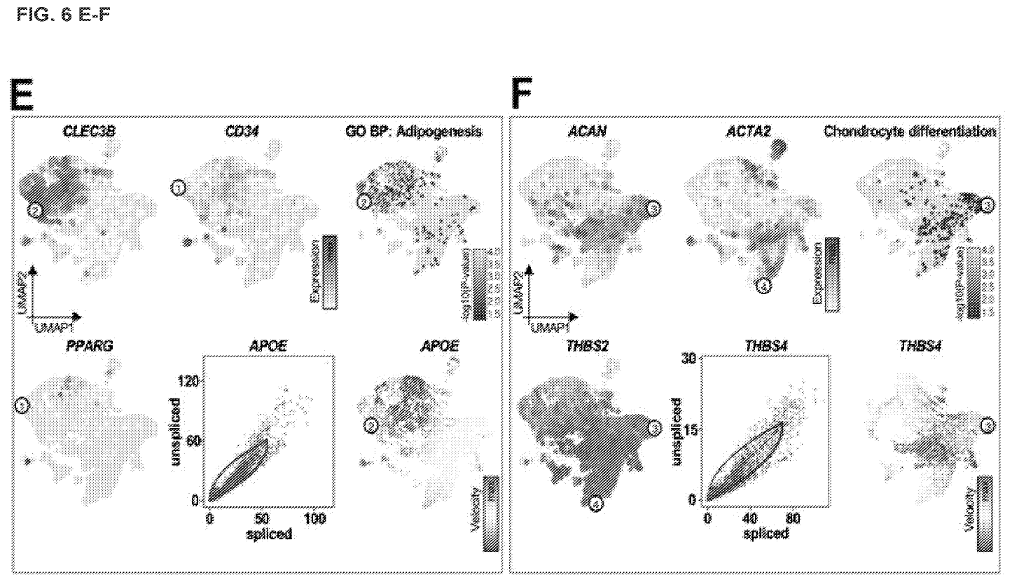

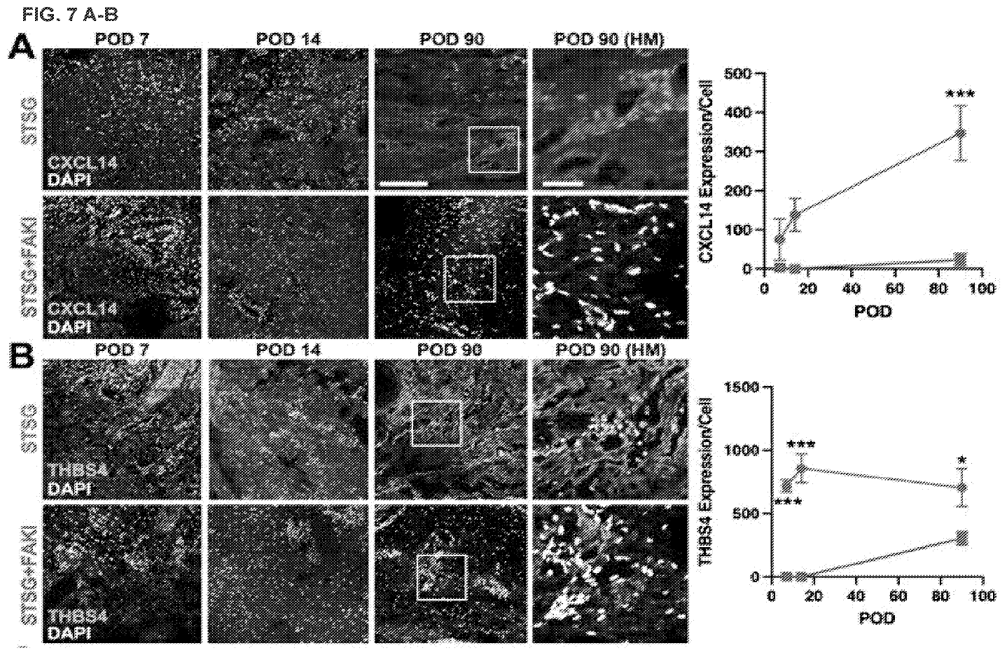

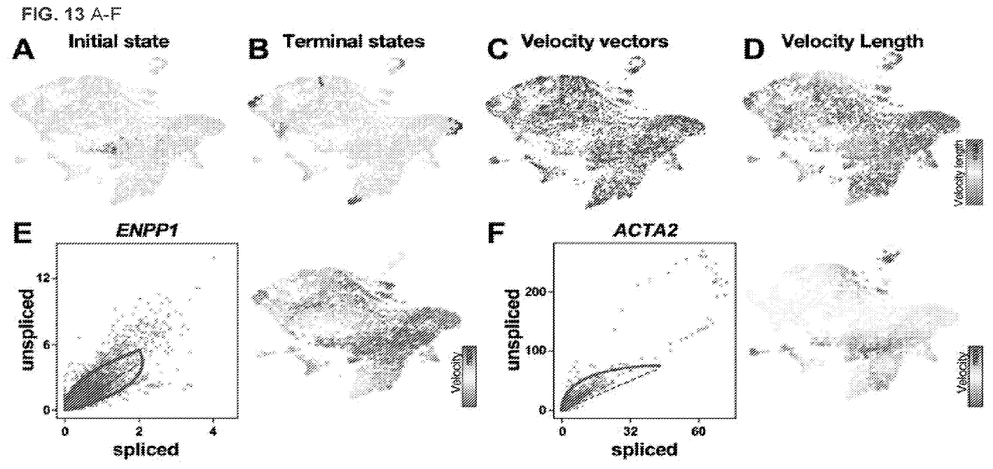

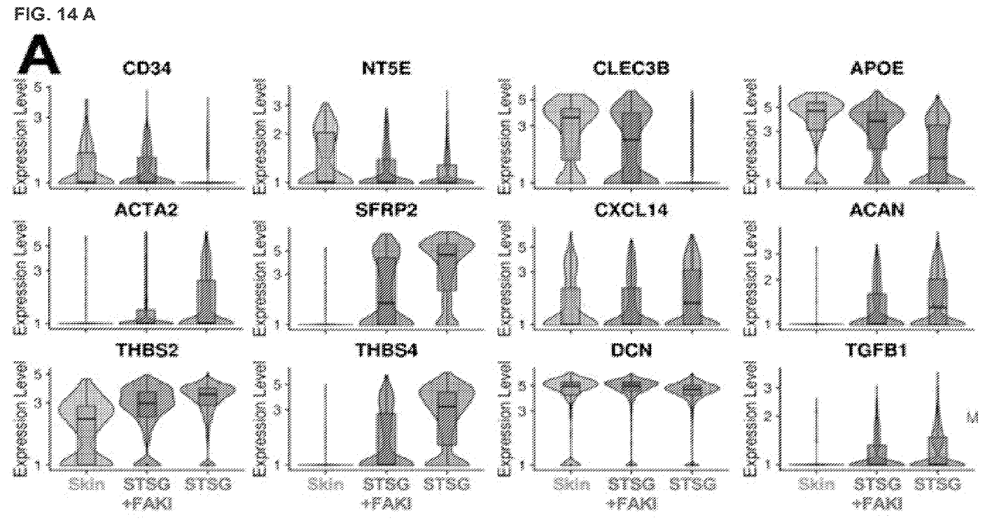

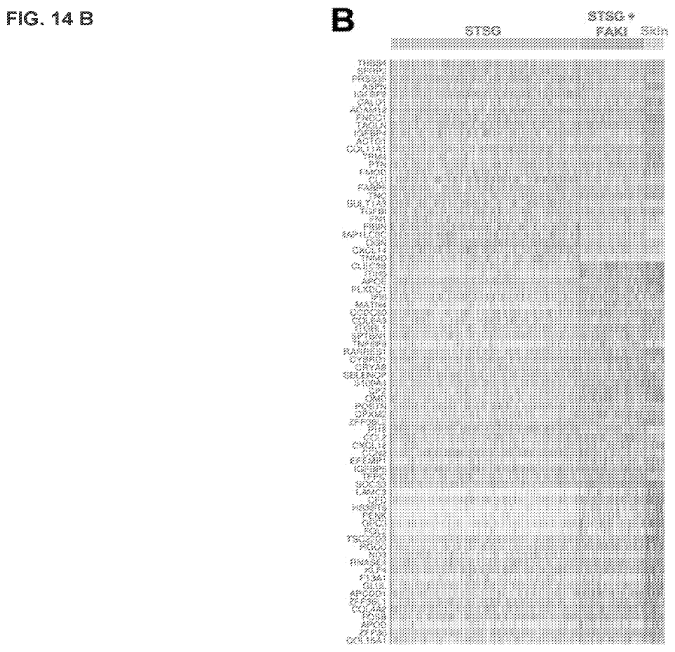

With these differences at early time points, we next sought to understand the role of mechanotransduction blockade at late time points. Because fibroblasts had the most gene changes between STSG scar and normal skin at day 90 (FIG. 2E), we specifically examined the effect of FAK inhibition on fibroblast differentiation states. First, we performed RNA velocity analysis using scVelo and CellRank (FIGS. 6, A and B), which combines RNA velocity information with transcriptomic similarity to compute a global map of cellular fate potentials uncovering initial and terminal cell states (FIG. 13A) (80, 81). CellRank identified six transcriptionally distinct cell lineages (FIG. 6B), which originated at the initial root state (labeled with *). Fibroblasts from FAKI-treated STSGs exhibited transcriptional similarity to those from unwounded skin, primarily in lineages 1 and 2, whereas cells from normal STSGs shifted away from this [Skin & STSG+FAKI] cell state along the UMAP-1 axis into four more heterogenous lineages (3,4,5, and 6) (FIGS. 6, A and B; FIG. 13, A to C). Fibroblast lineages 3 and 4 showed a higher latent time score and velocity vector length (FIG. 6C; FIG. 13D) indicating more advanced differentiation states with a higher proportion of mature spliced RNA. Thus, lineages 1,2,3, and 4 were selected for further analysis.

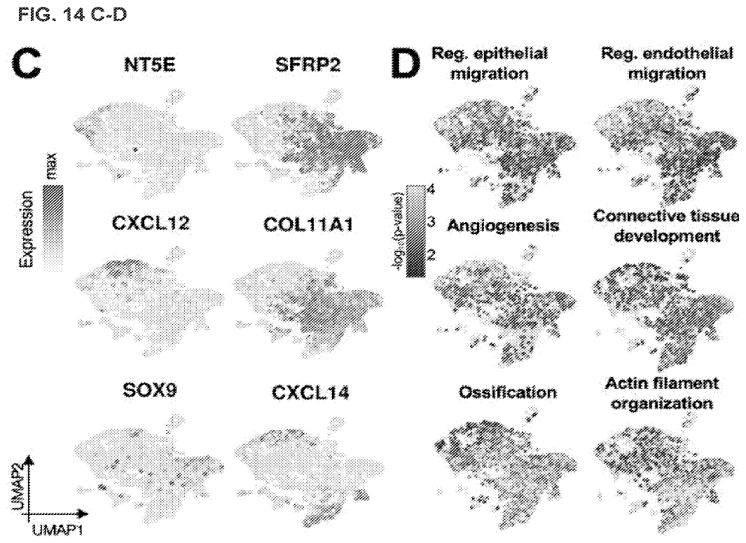

We combined fate probability estimates with a pseudotemporal ordering along latent time to visualize gene expression along trajectories leading to terminal states. This uncovered lineage drivers for the regenerative lineages 1 and 2, and the fibrotic lineages 3 and 4 (FIG. 6, D-F; FIGS. 13, A and B). Along the fibrotic state, we observed an upregulation of the fibrotic, chondrogenic markers THBS2 (thrombospondin 2; along states 3 and 4), THBS4 (state 3), ACAN (aggrecan; state 3), ENPP1 (ectonucleotide pyrophosphatase 1), as well as myofibroblast differentiation marker ACTA2 (state 4) (82-84), suggesting that mechanical forces push a subset of fibroblasts toward a more fibrotic fate during scar formation (FIG. 6, D to F; FIGS. 14, E and F). Aggrecan is a chondrocyte marker, while the family of THBS genes encode for thrombospondins, ECM proteins that facilitate cell-matrix binding and are known to modulate mesenchymal chondrogenic and adipogenic differentiation states (85). Comparison of the top DEGs in STSGs also revealed upregulation of previously identified pro-fibrotic genes, such as SFRP2 and TGFB1 (FIG. 14, A to C) (86, 87). SFRP2 has been previously identified in healthy human skin scRNA-seq as a fibroblast subpopulation with high fibrogenic potential (46-48), while transforming growth factor (TGF)β1 is a well-known promoter of fibrosis (88). This was supported by an enrichment for gene sets involved in contractile myofibroblast phenotypes (actin filament organization; lineage 3,4), chondrocyte differentiation (lineage 3), and ossification (lineage 3,4) (FIG. 6F; FIG. 14D). STSG fibroblasts also upregulated fibroblast-recruiting chemokine CXCL14 (FIG. 6F; FIG. 14, A to C). CXCL14 has been previously found to stimulate fibroblast migration and proliferation while inhibiting regenerative differentiation (89, 90).

By contrast, along regenerative lineages 1 and 2, FAKI-treated STSG and unwounded skin demonstrated similar expression of genes, showing an abrogation of the aforementioned pro-fibrotic genes as well as an upregulation of APOE (state 2), CLEC3B (state 2), CD34 (state 1), and PPARG (peroxisome proliferator-activated receptor gamma; state 1) unspliced pre-mRNA, indicating induction of regenerative, adipogenic transcription (91, 92) (FIGS. 6, D and E). Apolipoproteins, such as APOE and APOD, are key markers of lipid transport and lipid metabolism that are expressed in both lipid trafficking fibroblasts (lipofibroblasts) and adipocytes (92, 93). Apolipoproteins have been found to attenuate inflammation, and lipofibroblasts have been found to be a fibroblast subpopulation that interacts with adipose tissue and can differentiate into adipocytes during normal tissue healing (94). These lipofibroblasts also expressed PPARG and CFD (complement factor D, encoding for Adipsin), which also promote lipid accumulation and are critical transcription factors of adipogenesis (FIG. 6E). FAK inhibition in STSG (lineages 1 and 2) also promoted adipogenic gene sets (adipogenesis, angiogenesis, epithelial cell migration) and stem cell markers (CD34 and NT5E) (FIG. 6E; FIG. 14, B to D). The expression of these markers matched the expression seen in unwounded skin. Additionally, thrombospondins have been found to drive hypertrophic scar formation in a TGFβ1 dependent manner (95), and TGFβ1 has been found to inhibit adipogenesis (88). This down regulation of mechanotransduction, thrombospondins, and TGF was further supported by an upregulation of small leucine-rich proteoglycans (SLRPs) such as decorin (DCN) (FIG. 6D, FIG. 14A), which helps control the fibrillogenesis of scar formation, is highly expressed in unwounded skin compared to fibrotic tissue, and inhibits TGFβ1 to reduce HTS formation (87).

### 8. Mechanotransduction Modulates Temporal Evolution of Regenerative Fates

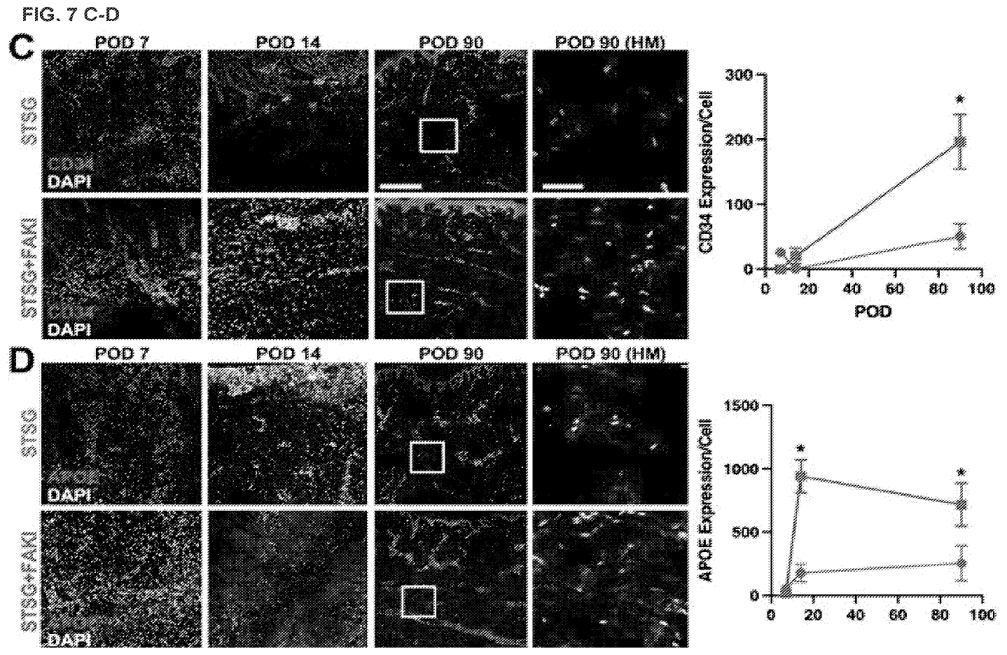

We then collected porcine STSG tissue across a range of time points (PODs 7, 14, 90) and mapped out a time course of fibroblast protein expression within the healing STSG tissue. Utilizing immunofluorescent staining, we found that untreated STSG demonstrated increased expression of fibroblast-recruiting chemokine CXCL14 at early time points, followed by significantly increased CXCL14 at late time points (POD90; ***P<0.001) (FIG. 7A). Furthermore, untreated cells expressed greater abundance of chondrogenic matrix protein thrombospondin 4 (protein form of THBS4) across POD 7 (***P<0.001), POD 14 (***P<0.001), and POD 90 (*P<0.05) (FIG. 7B). Thus, FAK inhibition attenuated THBS4 and CXCL14 expression (FIGS. 7, A and B), decreasing cellular recruitment and subsequent fibrotic matrix deposition. Disruption of mechanotransduction also initiated CD34 expression at late time points (FIG. 7C), demonstrating a shift of fibroblast transcriptional profiles toward more plastic, stem-like phenotypes. These stem-like fibroblasts subsequently demonstrated increased adipocyte lipid trafficking apolipoprotein E (protein form of APOE) at both PODs 14 (*P<0.05) and 90 (*P<0.05) (FIG. 7D), indicating an increased differentiation toward lipofibroblast, regenerative phenotypes.

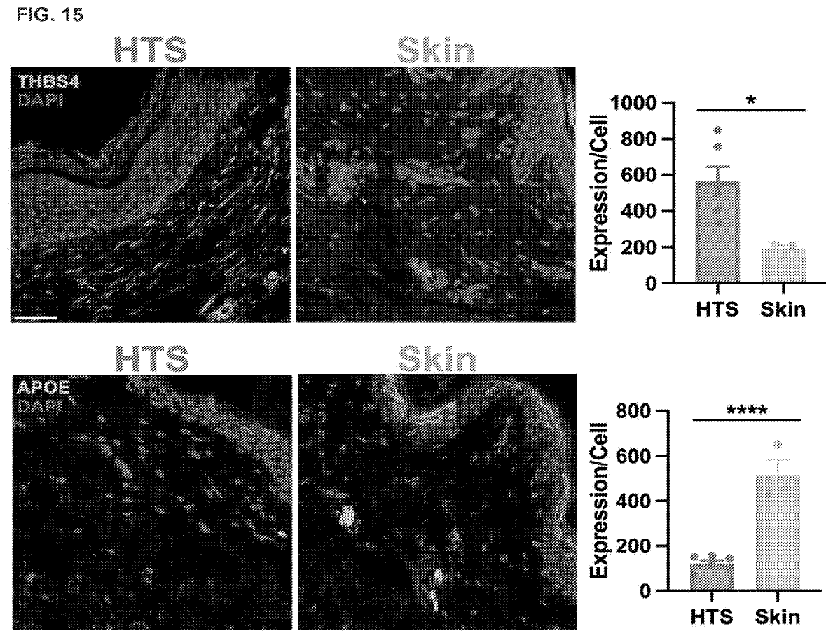

To confirm the potential human relevance of these findings, we collected patient samples from the clinic. Although we were unable to collect healed human STSG samples, we were able to collect human hypertrophic scars. We found that human hypertrophic scar demonstrated significantly increased thrombospondin 4 (*P<0.05) compared to unwounded skin, indicating an upregulation of extracellular matrix protein deposition in highly fibrotic scar tissue. Furthermore, healthy human skin demonstrated significantly increased apolipoprotein E (*P<0.05), suggesting the presence of healthy lipofibroblasts and adipocytes within the skin. These lipo-trafficking cells were not present within hypertrophic scar (FIG. 15).

Overall, these data demonstrated that FAK inhibition after injury promotes regenerative phenotypes and shifts fibroblasts away from fibrotic states. We identified a “regenerative axis” leading to the FAK-inhibited and unwounded skin clusters, as well as a “fibrotic axis” leading in the opposing direction and characterized by untreated STSG fibroblasts in a highly mechanically stressed environment. Next, we sought to recapitulate the existence of these two trajectories in a precisely controlled mechanical environment in vitro.

### 9. Precise Manipulation of Mechanical Forces Modulates Fibroblast Transcriptional Signatures

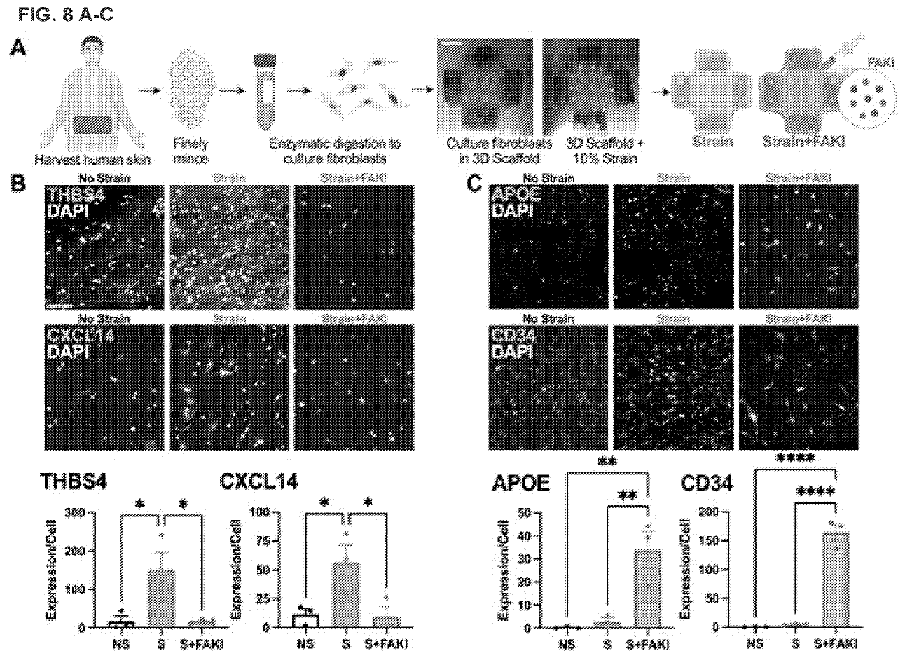

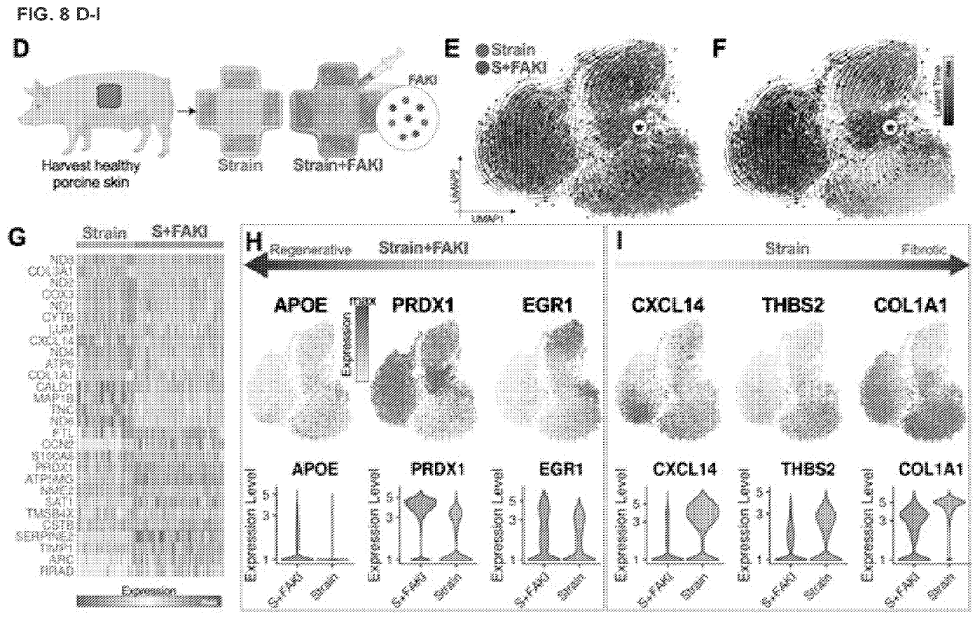

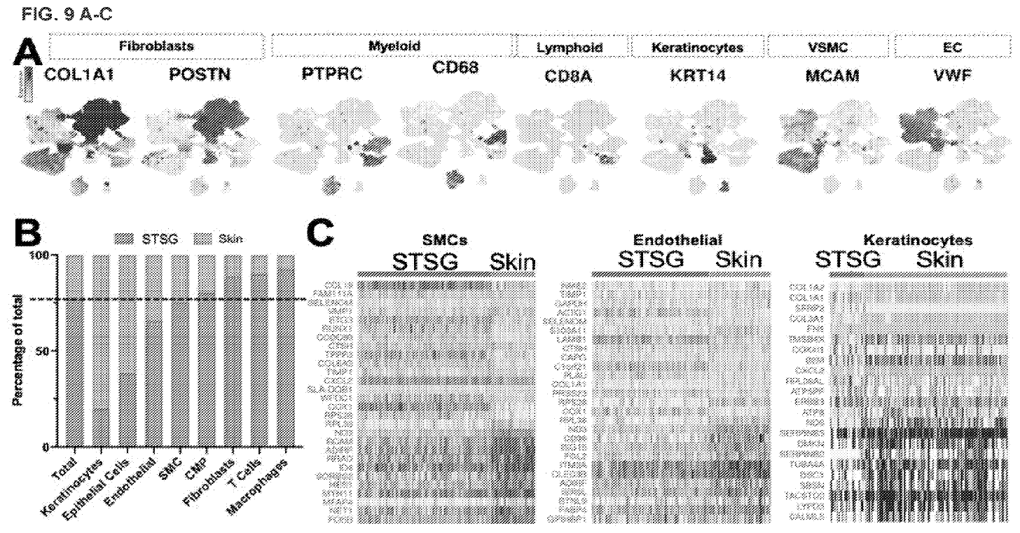

Our group and others have previously demonstrated that both porcine wounds as well as human skin are subjected to mechanical strains of around 5-20% (96, 97) and that physically offloading this mechanical tension with a physical bandage reduces fibrotic scar formation (38). To determine whether the increased mechanical strain experienced by cells within the healing STSG would trigger the same fibroblast transcriptional states observed in vivo, we used our previously published three-dimensional (3D) organotypic scar culture system that permits the precise manipulation of strain (and therefore stress) without any other potential confounding factors (27, 98). Fibroblasts were first isolated from human skin and seeded within 3D collagen scaffolds (FIG. 8A). These scaffolds were then subjected to either 10% strain (Strain, S), 10% strain with FAK inhibition (Strain+FAKI, S+FAKI), or 0% strain (No Strain, NS) as a control (FIG. 8, A to C). Because it seemed likely that healing tissue would not be subjected to maximal strains (due to pain), we used a strain profile of 10% to represent the average wound strain environment.

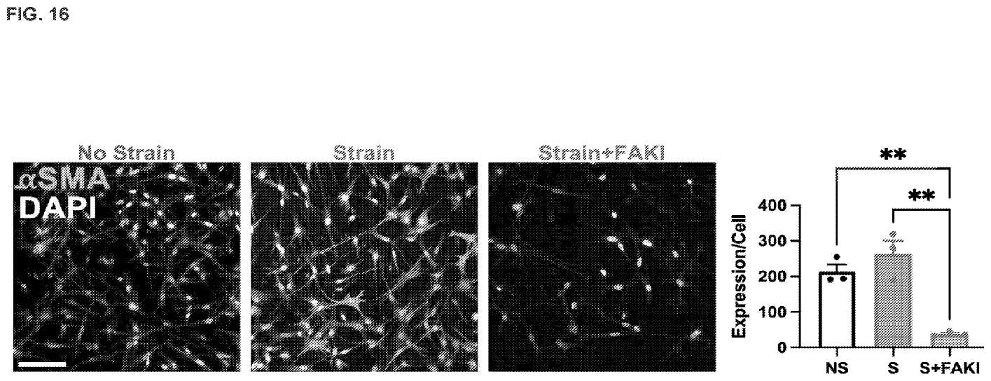

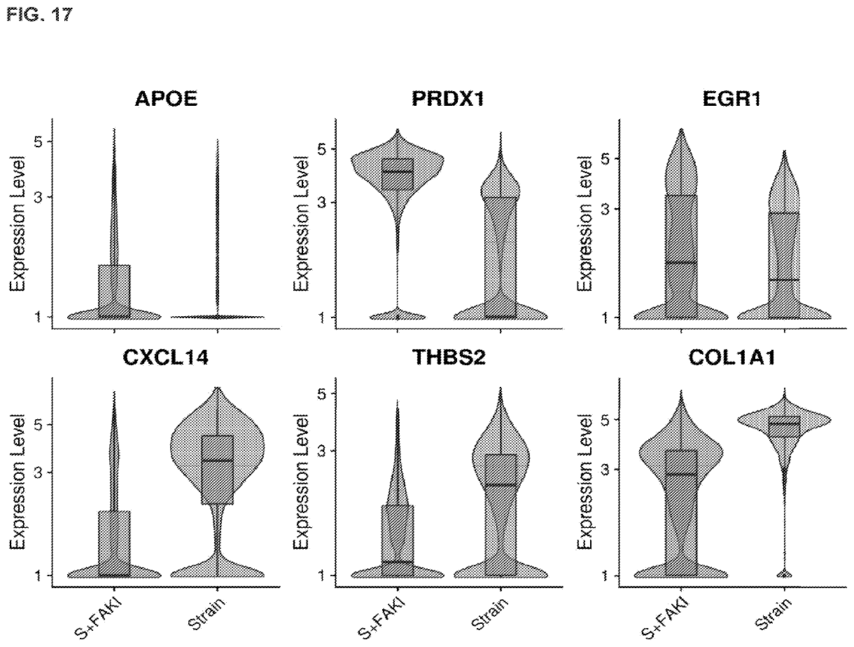

In human cells, we observed that mechanically strained fibroblasts upregulated both fibroblast-recruiting CXCL14 and fibrotic THBS4 expression (*P<0.05) compared to unstrained fibroblasts (FIG. 8B). Subsequent FAK inhibition of strained fibroblasts significantly decreased CXCL14 (*P<0.05), THBS4 (*P<0.05), and aSMA (**P<0.01) expression compared to strained fibroblasts (FIG. 8B; FIG. 16), while also significantly increasing APOE (**P<0.01) and CD34 (****P<0.0001) expression (FIG. 8C). These observations confirmed the beneficial transcriptional effects of FAKI on STSGs in vivo. Repeating these experiments using porcine cells and scRNA-seq, we then observed that strained fibroblasts demonstrated increased expression of collagen (COL1A1, COL3A1) and other ECM-related genes (FN1, ADAM12), as well as markers that have been found to drive fibrosis (including POSTN) (99) (FIG. 8, D to G). Utilizing RNA velocity, we again identified two differentiation trajectories radiating from the center of the embedding (indicated with a *) into either Strain (fibrotic) or S+FAKI (regenerative) lineages (FIGS. 8, E and F), matching our in vivo analysis (FIG. 6, A to C; FIG. 13). Also similar to our observations in vivo (FIG. 6C), strained fibroblasts were advanced in both latent time and velocity pseudotime, demonstrating large changes in transcription induced by mechanical strain (FIG. 8F). Strained fibroblasts upregulated CXCL14, fibrotic thrombospondin (THBS2), and collagen (COL1A1) expression and FAK inhibition abrogated all of these pro-fibrotic responses and instead demonstrated a regenerative transcriptomic signature characterized by an upregulation of APOE and the anti-fibrotic genes EGR1 (early growth response 1) and PRDX1 (peroxiredoxin 1) (FIGS. 8, H and I; FIG. 17), matching both our STSG (FIGS. 5 and 6) and human findings (FIG. 8, A to C). Overall, we observed that physiologic mechanical strain pushed both human and porcine fibroblasts toward the same transcriptional profiles seen in vivo, confirming that manipulating mechanical forces directly drive fibrotic and regenerative phenotypes in both large animals and humans.

Taken together, these findings characterize large animal and human scarring, demonstrating that mechanical forces promote fibroblasts to assume a distinct pro-fibrotic program, which may be averted and driven toward a regenerative commitment similar to unwounded skin by inhibiting mechanotransduction. Fibroblasts in healing STSGs are subjected to the natural high mechanical stress within skin tissue that subsequently activates mechanotransduction signaling and leads to contractile, chondrogenic differentiation and fibrotic collagen formation. In contrast, FAK inhibition pharmacologically shields fibroblasts from this high stress environment, preventing fibrotic phenotypes and promoting more regenerative, adipogenic phenotypes.

### 10. Discussion

Skin grafting is a mainstay for the treatment of severe burns and traumatic injuries and has revolutionized the survival of burn patients (8). Unfortunately, skin grafts are not a perfect replacement for injured skin and may result in debilitating fibrotic hypertrophic scar formation and severe contractures that may require multiple rounds of revision surgeries (8-12). In addition, there are currently no FDA-approved pharmacologic therapies to prevent these complications and improve outcomes after grafting (7, 13, 14). To understand the mechanisms driving STSG healing, we developed a clinically relevant large animal model to characterize the cellular milieu in STSG scar and unwounded skin and found that STSG exhibited sustained elevation of fibroproliferative and inflammatory transcriptional programs, driven by mechanically activated immune cells and myofibroblasts. Because mechanotransduction pathways were found to be upregulated, we judged mechanotransduction to be an appealing target to block excessive fibrosis and contracture following STSG.

Here, we demonstrate that sustained delivery of small molecules to modulate mechanotransduction through FAK inhibition could be used in conjunction with STSG to reduce scar contracture, promote regenerative dermal remodeling, and improve biomechanical skin properties in a human-like porcine model. We created this sustained release form within hydrogels, which are commonly employed in standard wound and skin graft care (100). This therapeutic is an effective therapy for human grafts that can be incorporated into the current standard of care, providing translational opportunities to help patients with traumatic and burn injuries.

To understand scar formation and healing, many groups, including our own, have comprehensively investigated the crucial role of the fibroblast in a wide range of fibrotic disease states (23, 25, 26, 32, 33, 101, 102). However, fibroblast transcriptional states have not previously been investigated in the context of large animal fibrosis or as part of skin graft healing. In the context of human scRNA-seq, several groups have previously characterized unwounded human skin (46-48) and compared human keloid to human scar without unwounded skin (49). However, direct comparisons of fibrotic tissue to unwounded skin or to pharmacologically enriched regenerative populations with single cell resolution have been lacking. In our study, we found that both myeloid cells and fibroblasts during late-stage scar formation had the largest transcriptional changes from cells in unwounded skin, and we identified that mechanical signaling in the STSG promotes differentiation into heterogenous, pro-fibrotic phenotypes. By disrupting mechanotransduction, we demonstrated the ability to divert mesenchymal fate commitment of both large animal and human fibroblasts away from these contractile myofibroblast phenotypes and toward adipogenic, regenerative differentiation fates, restoring the number of quiescent fibroblasts that produce low quantities of randomly organized collagen in unwounded skin.

We were surprised to identify that modulating mechanotransduction signaling could directly shift fibroblast differentiation between comparatively pro-fibrotic and pro-regenerative fates in vivo. Within various tissues, both mesenchymal progenitor cells (MPCs) and myofibroblasts, a cell type previously thought to be fully differentiated, have been found to be plastic cell types that can be pushed toward either fibrosis, osteo/chondrogenesis, or adipogenesis to differentiate into adipocytes (33, 103). Several previous in vitro studies have also investigated how changes in mechanical stimuli affect actin cytoskeletal organization and MPC differentiation. For example, McBeath et al. found that increased cellular mechanical stress guided adherent MPCs toward osteogenesis, whereas nonadherent, round MPCs were guided toward adipogenesis (104). Thrombospondins have also been found to promote osteo/chondrogenesis and inhibit adipogenesis in mesenchymal cells (105, 106). Here, we characterize these fibroblast fates on an “axis of fibrosis-regeneration,” with mechanical signaling pushing and pulling cells along this axis.

Unexpectedly, fibroblasts did not demonstrate large changes in transcriptional activity during the early stages of healing. Instead, at earlier time points, disruption of mechanotransduction primarily pushed myeloid-lineage cells toward CXCL10-mediated anti-inflammatory and regenerative states. Whereas previous studies focused on mechanoresponsive fibroblasts as the “drivers” of fibrosis (25), secreting chemokines to promote inflammatory cell recruitment (26), our findings revealed a more complicated interplay between immune cell and fibroblast mechanotransduction signaling during the different stages of healing. Controlling mechanical signaling in myeloid cells at early time points modulated secretion of inflammatory signals that could directly influence fibroblast phenotypes. Although some recent studies suggested a role for immune cells in regulating and sustaining these fibrotic processes (61-63), our findings reveal that myeloid cells may actually “drive” the fibroblasts. Many studies investigating other fibrotic disease states, such as renal fibrosis, idiopathic pulmonary fibrosis, non-alcoholic steatohepatitis (NASH), or liver cirrhosis have typically characterized the importance of each of these cell types in isolation. Based on our findings, future studies should interrogate the interplay between both cell types to improve therapeutic and translational outcomes for a wide range of fibrotic diseases states.

Collectively, our study represents a comprehensive characterization of fibrosis in both a human-scale model and human tissue, linking together the importance of modulating mechanical forces for tissue regeneration in both large organisms and humans. Pharmacological blockade of mechanotransduction could improve clinical outcomes by promoting early anti-inflammatory and late regenerative transcriptional programs to mitigate fibrosis and improve quality of life. Mitigating scar formation and promoting tissue regeneration in humans and other large organisms remains the “holy grail” of biomedical research (107), and our findings may have the potential to improve patient care for a number of fibrotic diseases. 20

### 11. Supplementary Materials and Methods:

a. Animal Care

All animal work was conducted in accordance with the Administrative Panel on Laboratory Animal Care protocols (APLAC #31530 and 32962) approved by Stanford University. Female red Duroc pigs, 6-8 weeks old and weighing approximately 16-20 kg at the time of surgery, were purchased from Pork Power Farms (Turlock, CA). All animals were acclimated for at least one week upon arrival. All animals were fed lab porcine grower diet and water ad lib.

b. Preparation of Animals for Surgery

Fasting of the animals was performed 12 hours prior to surgical procedures. Anesthesia was administered in cooperation with the Stanford Veterinary Service Center (VSC) personnel. Initially, the animals were sedated with administration of intramuscular telazol. A peripheral intravenous line was established in the vein of the ear. To minimize pain, local anesthetic in the form of EMLA (lidocaine 2.5% and prilocaine 2.5%) cream was applied over the vein prior to cannulation. During this time, 10 (mL/kg hr) of fluids was initiated. In accordance with institutional guidelines, general anesthesia was established with telazol 6-8 mg/kg IM administered once as a pre-anesthetic, and animals were intubated with an endotracheal tube and maintained on 1.5-3% of inhaled isoflurane throughout the procedure for maintenance of anesthesia. Animals were placed in ventral recumbency, and their backs were shaved. Skin was cleansed with Betadine solution and rinsed with alcohol to ensure no contamination from skin pathogens. Animals were draped in a sterile fashion to maintain a sterile field. Heat support and eye lubrication were also used. Each surgical procedure lasted approximately 2 hours once the animals were sedated.

To prevent surgical site infection, antibiotics were given prophylactically and post-operatively. Cefazolin 25 mg/kg IV was given 60 minutes prior to the initial insult and repeated at 12 hours after the end of the surgery. Oral amoxicillin 10 mg/kg was given twice a day for one week. Wounds were wrapped in the appropriate dressings. Control of pain was achieved by the administration of transdermal fentanyl 50 mcg/h 24 hours in advance of surgery. Hydromorphone (0.05 mg/kg) IM was used if the fentanyl patch came off or was not placed prior to surgery. Carprofen was used once post-operatively, followed by once every 24 hours for 1-2 days, then as needed based on pain assessment.

c. Blank and FAKI-Releasing Pullulan-Collagen Hydrogel Production

We produced blank and FAKI-releasing hydrogels as previously described (23) with some modifications. 1 g pullulan (TCI 9057-02-7, Tokyo) was mixed with 1 g of trisodium trimetaphosphate (STMP) (Sigma Aldrich T5508) and 1 g potassium chloride (KCl) (Fisher Scientific 7447-40-7). The powder was mixed with deionized water to a total volume of 5 mL and thoroughly mixed, followed by 5 mL of 10 mg/mL bovine collagen suspension in 0.01M hydrochloric acid (HCl). The resulting mixture was vortexed until homogenous and then 0.65 mL of 1N sodium hydroxide NaOH to initiate crosslinking was also added with gentle vortexing. The mixture was poured into silicon molds and then allowed to dry overnight in a sterile hood at room temperature. Dried films were washed with deionized water to remove non-crosslinked polymers, NaOH, and KCl. The pH of the wash solution was measured and continually washed until it reached a pH between 7.0 to 7.5. The swollen hydrogels were frozen at −80° C. before lyophilization to produce dry (blank) patches.

FAKI compound was obtained from Verastem Oncology (VS-6062) and Selleckchem (625249). We dissolved FAKI in acetone at 1 mg/ml; 1 mL of the solution was poured uniformly on the porous hydrogel and then allowed to evaporate the solvent in a sterile hood. Blank and FAKI-containing hydrogel patches were placed in individual plastic bags and sterilized using e-beam irradiation at a 20 kGy irradiation dose.

d. Patch FAKI Release Studies

In the pullulan-collagen hydrogel used in our current studies, FAKI molecules are physically encapsulated in the hydrogel pores. We conducted in vitro release studies of FAKI from this scaffold and demonstrated that controlled and sustained release of FAKI continues up to 72 hours. Briefly, we took three two blank hydrogels and three FAKI hydrogels and placed them into a 15,000 or 25,000 MWCO dialysis membrane tube (Thermo Fisher Scientific). Then, we added 0.5 mL of PBS to swell the hydrogel inside the dialysis tube, sealed the dialysis tube on both sides, and then immersed the tubes into 10 mL of PBS in a 50 ml Falcon tube. We placed the tube to shake at 37° C. and moved the sample into a new tube with fresh PBS at each of the following time points: 2h, 4h, 6h, 8h, 24 h, 96h. We then analyzed the FAKI content of the collected 5 samples with LCMS (Applied Biosystems, API 4000 Q Trap) to calculate the FAKI content in each patch (FIG. 10B). To study release on porcine skin, we placed FAKI patches (2 mg FAKI/25 cm²) on porcine skin for 12 and 24 hours to determine time-dependent release. FAKI was detected as deep as 2 mm from the stratum corneum when treated for 24 hours (FIG. 10C).

e. Scar Contracture Analysis

STSG were monitored photographically at each dressing change. Each image was taken with a ruler or standard measurement device to standardize measurements. Scar areas were traced over time in ImageJ. Contracture was measured as change in scar area, normalized to the area of the STSG during the first dressing change. Two independent evaluators measured scar area, and the resultant scar area was the average of the two measurements.

f. Visual Scar and Interstitial Epithelialization Assessment

Gross photographs of each wound were taken at each dressing change. Quantification of scar metrics were performed with these gross photographs by a panel of three blinded scar experts using a Visual Analog Scale (VAS) for 5 components: vascularity, pigmentation, observer comfort (e.g., overall cosmesis), acceptability, and contour. Total scores were calculated as a composite of all 5 scores; lower scores indicate improved scar appearance. Blinded experts also assessed the amount of interstitial epithelialization, defined by the amount of re-epithelialization between the interstitial spaces of the meshes in the graft over time. Scoring was also performed for unwounded skin as an additional control.

g. Viscoelastic Analysis of Skin Biomechanical Properties

A cutometer (Dual MPA 580, Courage+Khazaka Electronic) was used to evaluate the firmness and elasticity of the healing tissue and unwounded skin. Cutometer assessment is one of the most common instruments to measure viscoelasticity in human patients (111, 112), measuring the vertical deformation of the skin surface by applying negative pressure (suction) through a small circular diameter (8 mm probe). Deformation (suction) for two seconds followed by two seconds of relaxation (no suction) was applied three times and averaged. The firmness 0 was measured as the amplitude at the end of the suction phase (RO metric) and normalized against the values of STSG treated with blank hydrogels (111). Increased deformation to a consistent negative pressure corresponds to less firm tissue, more similar to unwounded skin.

h. Collection and Cryo-Sectioning of Human and Porcine Tissue Specimens

Porcine specimens were harvested from the center of each wound at the end of the study. Human hypertrophic scar (HTS) and unwounded skin samples were obtained under the approved IRB (#54225). The tissue samples collected from the study would otherwise be discarded. No patient identifying information was retained with the samples. These specimens were immediately fixed in 4% paraformaldehyde, dehydrated, and cryo-embedded in optimal cutting temperature (OCT) compound for frozen sectioning on a microtome-cryostat.

i. Histological Analysis of Collagen Architecture

Masson's Trichrome staining and Picrosirius Red staining were performed, and images were captured with a Leica DM5000 B upright microscope. For Picrosirius Red, polarized light microscopy was used to obtain 40× magnification images and analysis of fiber alignment was performed using CurveAlign (109) and MatFiber (98, 110). The strength of alignment ranges from a value of 0 (completely random fiber alignment) to 1 (completely aligned fibers). Quantification of individual collagen fiber parameters (fiber length, width) was performed using CT-FIRE (http://loci.wisc.edu/software/ctfire) (109, 113).

j. Immunofluorescent Staining

Immunofluorescent staining was performed using primary antibodies targeting CXCL10 (Thermo Fisher Scientific, PA5-46999), F4/80 (Thermo Fisher Scientific, MF48000), CXCL14 (Thermo Fisher Scientific, 10468-1-AP), Thrombospondin 4 (THBS4) (Abcam, ab263898), Apolipoprotein E (APOE) (Abcam, ab52607), CD34 (Abcam, ab81289), and alpha smooth muscle actin (aSMA) (Abcam, ab5694). The amount of fluorescent area was quantified and normalized to the number of cells (individual DAPI nuclei) using a custom MATLAB image processing code written by the authors and previously published (98). All histology and immunofluorescent images shown are representative images of multiple experiments.

k. Single Cell Barcoding, Library Preparation, and Single Cell RNA Sequencing

We obtained a 1 cm² tissue from the center of our STSG groups or unwounded skin. The tissue was thoroughly cut into small 1 mm² pieces using a scalpel before then being carefully minced with fine scissors. To maximize cell capture for scRNA-seq, diligent care was made to cut the tissue into a fine tissue paste consistency. An increased enzyme concentration and volume of 30 ml of 1 mg/mL Liberase (Sigma-Aldrich 5401127001) in PBS were used to fully break down the dense, fibrotic porcine extracellular matrix. The cell-digest suspensions were constantly agitated (rotated) for a total of 2 hours at 37° C. Tissue solution was subjected to maximum speed vortex mixer (VWR) for 30 seconds before being placed in the oven, after 1 hour, and again after total of 2 hours to physically disrupt any tissue that had clumped together and maximize the tissue surface area exposed to enzymatic digestion at all times.

The tissue solution was filtered through a 100 μm Nylon cell filter (Fisher-Scientific 08-771-19) into a new conical tube, and 20 mL of 10% FBS DMEM was added through the filter to quench the enzymatic reaction and release any cells trapped within the filter, maximizing downstream cell yield. Solutions were spun at 350×g for 5 min at 4° C. in a centrifuge, supernatant was aspirated, and cells were then resuspended in 20 mL 10% FBS DMEM and passed through a 70 μm Nylon cell filter. A 20 mL solution of 10% FBS in PBS (FACS Buffer) was added through the filter to wash the remaining cells.

This cellular suspension was then resuspended in a concentrated solution and submitted for droplet-based microfluidic single cell RNA sequencing (scRNA-seq) at the Stanford Functional Genomics Facility (SFGF) using the 10× Chromium Single Cell platform (Single Cell 3′ v3, 10× Genomics, USA). The cell suspension, reverse transcription master mix, and partitioning oil was loaded onto a single cell chip, processed on the Chromium Controller, and reverse transcription was performed at 53° C. for 45 min. cDNA was amplified for 12 cycles total (BioRad C1000 Touch thermocycler) with cDNA size selected using SpriSelect beads (Beckman Coulter, USA) and a 3:5 ratio of SpriSelect reagent volume to sample volume. cDNA was analyzed on an Agilent Bioanalyzer High Sensitivity DNA chip for qualitative control, fragmented for 5 min at 32° C., followed by end repair and A-tailing at 65° C. for 30 min, and then double-sided size selected with SpriSelect. Sequencing adaptors were ligated to the cDNA at 20° C. for 15 min. cDNA was amplified using a sample-specific index oligo as primer, followed by another round of double-sided size selection. Final libraries were analyzed on an Agilent Bioanalyzer High Sensitivity DNA chip for qualitative control purposes. cDNA libraries were sequenced on a HiSeq 4000 Illumina platform aiming for 50,000 reads per cell.

l. Custom Pig Transcriptome

We created a custom pig transcriptome generated using the Sus scrofa genome (Ensemble, v11.1) in accordance with 10× protocols for non-model organisms (44), and we have made it publicly available (see Data Availability and Materials) with instructions to implement as part of a modified 10× CellRanger pipeline. Briefly, the swine reference genome (Sus scrofa 11.1, release 98) was obtained from the Ensembl web server (https://uswest.ensembl.org/Sus_scrofa/Info/Index). Fasta files for individual chromosomes were merged using batch concatenation in cshell. GTF annotations were filtered using the CellRanger (10× Genomics) “mkgtf” function using the following arguments:

| | |
|---|---|
| --attribute=gene_biotype:protein_coding \ | --attribute=gene_biotype:lincRNA \ |
| --attribute=gene_biotype:antisense \ | -- |

attribute=gene_biotype:IG_LV_gene \

| | |
|---|---|
| --attribute=gene_biotype:IG_V_pseudogene \ | -- |

attribute=gene_biotype:IG_V_gene \

| | |
|---|---|
| --attribute=gene_biotype:IG_J_pseudogene \ | -- |

attribute=gene_biotype:IG_J_gene \

| | |
|---|---|
| --attribute=gene_biotype:IG_C_pseudogene \ | -- |

attribute=gene_biotype:IG_C_gene \

| | |
|---|---|
| --attribute=gene_biotype:TR_V_pseudogene \ | -- |

attribute=gene_biotype:TR_V_gene \

| | |
|---|---|
| --attribute=gene_biotype:TR_J_pseudogene \ | -- |

attribute=gene_biotype:TR_J_gene \

| | |
|---|---|
| --attribute=gene_biotype:TR_D_gene \ | -- |

attribute=gene_biotype:TR_C_gene
--attribute=gene_biotype:IG_D_gene \

These GTF and FASTQ files were then assembled using the CellRanger “mkref” command with default parameterization. All read fragments were aligned to this transcriptome using standard base-matching thresholds (51), followed by cell demultiplexing and UMI batch correction (52), allowing us to generate single cell mRNA count matrices for each pig sample.

m. Data Processing, FASTQ Generation, and Read Mapping

Base calls were converted to reads using the Cell Ranger (10× Genomics; version 3.1) implementation of mkfastq and then aligned against our custom pig transcriptome using Cell Ranger's count function with SC3Pv3 chemistry and 5,000 expected cells per sample (51). Cell barcodes were filtered for high quality based on an optimized threshold of having at least 200 unique transcripts profiled, less than 10,000 total transcripts, and less than 10% of their transcriptome of mitochondrial origin (114).

n. Data Normalization and Generation of Characteristic Subpopulation Markers

Unique molecular identifiers (UMIs) from each cell barcode were retained for all downstream analysis. Raw UMI counts were normalized with a scale factor of 10,000 UMIs per cell and subsequently natural log transformed with a pseudocount of 1 using the R package Seurat (version 3.1.1) (52). Highly variable genes were identified, and cells were scaled by regression to the fraction of mitochondrial transcripts. Aggregated data was then evaluated using uniform manifold approximation and projection (UMAP) analysis over the first 15 principal components (115). Automated cell annotations were ascribed using the SingleR toolkit (version 3.11) against the ENCODE blue database (116). Since we utilized a human annotation, we confirmed the cell types by also looking at cell-specific markers within our dataset. Differentially expressed genes were identified with Seurat's native FindMarkers function with a log fold change threshold of 0.5 using the ROC test to assign predictive power to each gene.

o. Over-Representation Analysis Using Genetrail3

Using Genetrail 3 (53), an over-representation analysis (ORA) was performed for each cell using the 500 most expressed protein coding genes with the gene sets Gene Ontology: 0.5 Biological Process (GO-BP) and WikiPathways (WP). P-values were adjusted using the Benjamini-Hochberg procedure and gene sets were required to have between 2 and 1000 genes.

p. RNA Velocity Analysis Using scVelo

RNA velocity analysis was performed using the dynamical model of the scVelo package(80). Partition-based graph abstraction (PAGA) was performed using the sc.tl.paga function in scVelo.

To find genes with differentially regulated transcriptional dynamics compared to all other clusters, a Welch t-test with overestimated variance to be conservative was applied using the sc.tl.rank_velocity_genes function. Genes were ranked by their likelihood obtained from the dynamical model grouped by treatment.

q. CellRank

CellRank was used to uncover the dynamic process of fibroblast differentiation based on Markov state modeling of single-cell data. Initial and terminal cell states as well as fibroblast fate probabilities were computed with CellRank based on dynamical RNA velocity information provided by scVelo.

r. Fibroblast-Populated 3D Collagen Scaffold Experiments

We isolated and cultured dermal fibroblasts from both healthy human and porcine skin samples. Fibroblasts were isolated by mechanical and enzymatic digestion and cultured under standard conditions until passage 3. Following our previously published protocols (98), we used the primary fibroblasts to create fibroblast-populated collagen hydrogels with a concentration of 200 k cells/mL and 2 mg/mL collagen (PureCol, Advanced Biomatrix 5005). In brief, collagen scaffolds were formulated in a cruciform shape with sponges in the arms and cultured in petri dishes on top of a layer (˜0.5 cm) of cured polydimethylsiloxane (PDMS; Sylgard 184 Silicone Elastomer Kit; Dow Corning). Pins were pushed through the sponges in the hydrogel cruciform arms to constrain the scaffolds during an initial pre-culture period (24h). Then, we subjected the scaffolds to either 0% strain (no strain) or 10% equibiaxial strain (either with or without FAKI) for an additional 48 hours. Strained but untreated cells were given 10 μL DMSO in 20 mL culture media, while strained and treated cells were administered 10 μL of 20 mM FAKI in DMSO in 20 mL culture media to achieve a final concentration of 10 UM FAKI.

We used a suture to paint nine Titanium(IV) oxide dots (Sigma-Aldrich 248576) on the surface of the central region of the gel to track and quantify the imposed strains. We used a digital camera to image the markers before and after strain. Strain was imposed by removing the pins, manually extending the hydrogel cruciform arms, and pushing the pins back through the arms to hold the scaffold in a new, extended position. Photographs of the marker positions were used to compute a single homogenous deformation gradient tensor F that provided the least-squares best fit mapping of the 9 marker positions from the undeformed to deformed positions by solving the overdetermined matrix equation:

$$x = FX + p , \qquad [ 1 ]$$

where p is an arbitrary vector included to account for translation between images. We converted the deformation to a strain tensor E using:

$$E = \frac{1}{2} ( {[ {F}^{T} F ]}^{2} - I ) \qquad [ 2 ]$$

where I is a 3×3 identity matrix with ones on the main diagonal and zeros elsewhere, and F^{T} represents the transpose of the matrix, according to linear algebra principles (117). By calculating this strain matrix E, we determined the strain in both the x and y directions. We manually adjusted the pins and extended the arms iteratively, taking additional pictures and making subsequent strain calculations until the overall strain imposed in both directions was around 0.10 (±0.01).

### II. REFERENCES

1. Z. Zhu, J. Ding, E. E. Tredget, The molecular basis of hypertrophic scars. Burns Trauma 4, 2 (2016).
2. R. K. V. Sethi, E. D. Kozin, P. J. Fagenholz, D. J. Lee, M. G. Shrime, S. T. Gray, Epidemiological Survey of Head and Neck Injuries and Trauma in the United States. Otolaryngology—Head and Neck Surgery 151, 776-784 (2014).
3. T. A. Wynn, T. R. Ramalingam, Mechanisms of fibrosis: therapeutic translation for fibrotic disease. Nat Med 18, 1028-1040 (2012).
4. B. Berman, M. H. Viera, S. Amini, R. Huo, I. S. Jones, Prevention and Management of Hypertrophic Scars and Keloids After Burns in Children. Journal of Craniofacial Surgery 19, 989-1006 (2008).
5. C. Mock, M. Peck, M. Peden, E. Krug, W. H. Organization, A WHO plan for burn prevention and care. Geneva: World Health Organization 3, (2008).
6. L. Deeter, M. Seaton, G. J. Carrougher, K. McMullen, S. P. Mandell, D. Amtmann, N. S. Gibran, Hospital-acquired complications alter quality of life in adult burn survivors: report from a burn model system. Burns: journal of the International Society for Burn Injuries 45, 42-47 (2019).
7. A. W. Chua, Y. C. Khoo, B. K. Tan, K. C. Tan, C. L. Foo, S. J. Chong, Skin tissue engineering advances in severe burns: review and therapeutic applications. Burns Trauma 4, 3 (2016).
8. L. F. Rose, J. C. Wu, A. H. Carlsson, D. I. Tucker, K. P. Leung, R. K. Chan, Recipient wound bed characteristics affect scarring and skin graft contraction. Wound repair and regeneration: official publication of the Wound Healing Society [and] the European Tissue Repair Society 23, 287-296 (2015).
9. J. D. Villapalos, P. vol. 2019.
10. R. Kandiyali, H. Thom, A. E. Young, R. Greenwood, N. J. Welton, Cost-effectiveness and value of information analysis of a low-friction environment following skin graft in patients with burn injury. Pilot Feasibility Stud 6, 8 (2020).
11. M. Singh, K. Nuutila, K. C. Collins, A. Huang, Evolution of skin grafting for treatment of burns: Reverdin pinch grafting to Tanner mesh grafting and beyond. Burns: journal of the International Society for Burn Injuries 43, 1149-1154 (2017).
12. T. Nuri, K. Ueda, Y. Fujimori, Ten-year Follow-up After Treating Extended Burn Scar Contracture with an Autologous Cultured Dermal Substitute. Plast Reconstr Surg Glob Open 6, e1782 (2018).
13. K. Meier, L. B. Nanney, Emerging new drugs for scar reduction. Expert Opin Emerg Drugs 11, 39-47 (2006).
14. L. Block, A. Gosain, T. W. King, Emerging Therapies for Scar Prevention. Adv Wound Care (New Rochelle) 4, 607-614 (2015).
15. D. Duscher, Z. N. Maan, V. W. Wong, R. C. Rennert, M. Januszyk, M. Rodrigues, M. Hu, A. J. Whitmore, A. J. Whittam, M. T. Longaker, G. C. Gurtner, Mechanotransduction and fibrosis. J Biomech 47, 1997-2005 (2014).
16. R. S. Gieni, M. J. Hendzel, Mechanotransduction from the ECM to the genome: are the pieces now in place? J Cell Biochem 104, 1964-1987 (2008).
17. N. Wang, J. D. Tytell, D. E. Ingber, Mechanotransduction at a distance: mechanically coupling the extracellular matrix with the nucleus. Nat Rev Mol Cell Biol 10, 75-82 (2009).
18. G. C. Gurtner, S. Werner, Y. Barrandon, M. T. Longaker, Wound repair and regeneration. Nature 453, 314-321 (2008).
19. S. Aarabi, K. A. Bhatt, Y. Shi, J. Paterno, E. I. Chang, S. A. Loh, J. W. Holmes, M. T. Longaker, H. Yee, G. C. Gurtner, Mechanical load initiates hypertrophic scar formation through decreased cellular apoptosis. FASEB J 21, 3250-3261 (2007).
20. A. Zehender, J. Huang, A. H. Gyorfi, A. E. Matei, T. Trinh-Minh, X. Xu, Y. N. Li, C. W. Chen, J. Lin, C. Dees, C. Beyer, K. Gelse, Z. Y. Zhang, C. Bergmann, A. Ramming, W. Birchmeier, O. Distler, G. Schett, J. H. W. Distler, The tyrosine phosphatase SHP2 controls TGFbeta-induced STAT3 signaling to regulate fibroblast activation and fibrosis. Nat Commun 9, 3259 (2018).
21. D. S. Foster, C. D. Marshall, G. S. Gulati, M. S. Chinta, A. Nguyen, A. Salhotra, R. E. Jones, A. Burcham, T. Lerbs, L. Cui, M. E. King, A. L. Titan, R. C. Ransom, A. Manjunath, M. S. Hu, C. P. Blackshear, S. Mascharak, A. L. Moore, J. A. Norton, C. J. Kin, A. A. Shelton, M. Januszyk, G. C. Gurtner, G. Wernig, M. T. Longaker, Elucidating the fundamental fibrotic processes driving abdominal adhesion formation. Nat Commun 11, 4061 (2020).
22. V. W. Wong, K. C. Rustad, S. Akaishi, M. Sorkin, J. P. Glotzbach, M. Januszyk, E. R. Nelson, K. Levi, J. Paterno, I. N. Vial, A. A. Kuang, M. T. Longaker, G. C. Gurtner, Focal adhesion kinase links mechanical force to skin fibrosis via inflammatory signaling. Nat Med 18, 148-152 (2011).
23. K. Ma, S. H. Kwon, J. Padmanabhan, D. Duscher, A. A. Trotsyuk, Y. Dong, M. Inayathullah, J. Rajadas, G. C. Gurtner, Controlled Delivery of a Focal Adhesion Kinase Inhibitor Results in Accelerated Wound Closure with Decreased Scar Formation. J Invest Dermatol 138, 2452-2460 (2018).
24. Q. Ding, C. L. Gladson, H. Wu, H. Hayasaka, M. A. Olman, Focal adhesion kinase (FAK)-related non-kinase inhibits myofibroblast differentiation through differential MAPK activation in a FAK-dependent manner. J Biol Chem 283, 26839-26849 (2008).
25. S. Mascharak, H. E. desJardins-Park, M. F. Davitt, M. Griffin, M. R. Borrelli, A. L. Moore, K. Chen, B. Duoto, M. Chinta, D. S. Foster, A. H. Shen, M. Januszyk, S. H. Kwon, G. Wernig, D. C. Wan, H. P. Lorenz, G. C. Gurtner, M. T. Longaker, Preventing Engrailed-1 activation in fibroblasts yields wound regeneration without scarring. Science 372, (2021).
26. V. W. Wong, K. C. Rustad, S. Akaishi, M. Sorkin, J. P. Glotzbach, M. Januszyk, E. R. Nelson, K. Levi, J. Paterno, I. N. Vial, A. A. Kuang, M. T. Longaker, G. C. Gurtner, Focal adhesion kinase links mechanical force to skin fibrosis via inflammatory signaling. Nature medicine 18, 148-152 (2011).
27. K. Chen, S. H. Kwon, D. Henn, B. A. Kuehlmann, R. Tevlin, C. A. Bonham, M. Griffin, A. A. Trotsyuk, M. R. Borrelli, C. Noishiki, J. Padmanabhan, J. A. Barrera, Z. N. Maan, T. Dohi, C. J. Mays, A. H. Greco, D. Sivaraj, J. Q. Lin, T. Fehlmann, A. M. Mermin-Bunnell, S. Mittal, M. S. Hu, A. I. Zamaleeva, A. Keller, J. Rajadas, M. T. Longaker, M. Januszyk, G. C. Gurtner, Disrupting biological sensors of force promotes tissue regeneration in large organisms. Nat Commun 12, 5256 (2021).
28. R. D. Galiano, J. t. Michaels, M. Dobryansky, J. P. Levine, G. C. Gurtner, Quantitative and reproducible murine model of excisional wound healing. Wound repair and regeneration: official publication of the Wound Healing Society [and] the European Tissue Repair Society 12, 485-492 (2004).
29. N. Naldaiz-Gastesi, O. A. Bahri, A. López de Munain, K. J. A. McCullagh, A. Izeta, The panniculus carnosus muscle: an evolutionary enigma at the intersection of distinct research fields. J Anat 233, 275-288 (2018).
30. C. P. Denton, P. A. Merkel, D. E. Furst, D. Khanna, P. Emery, V. M. Hsu, N. Silliman, J. Streisand, J. Powell, A. Akesson, J. Coppock, F. Hoogen, A. Herrick, M. D. Mayes, D. Veale, J. Haas, S. Ledbetter, J. H. Korn, C. M. Black, J. R. Seibold, Recombinant human anti-transforming growth factor beta1 antibody therapy in systemic sclerosis: a multicenter, randomized, placebo-controlled phase I/II trial of CAT-192. Arthritis and rheumatism 56, 323-333 (2007).
31. D. M. DeBruler, B. N. Blackstone, K. L. McFarland, M. E. Baumann, D. M. Supp, J. K. Bailey, H. M. Powell, Effect of skin graft thickness on scar development in a porcine burn model. Burns: journal of the International Society for Burn Injuries 44, 917-930 (2018).
32. C. F. Guerrero-Juarez, P. H. Dedhia, S. Jin, R. Ruiz-Vega, D. Ma, Y. Liu, K. Yamaga, O. 32. Shestova, D. L. Gay, Z. Yang, K. Kessenbrock, Q. Nie, W. S. Pear, G. Cotsarelis, M. V. Plikus, Single-cell analysis reveals fibroblast heterogeneity and myeloid-derived adipocyte progenitors in murine skin wounds. Nature Communications 10, 650 (2019).
33. M. V. Plikus, C. F. Guerrero-Juarez, M. Ito, Y. R. Li, P. H. Dedhia, Y. Zheng, M. Shao, D. 33 L. Gay, R. Ramos, T.-C. Hsi, J. W. Oh, X. Wang, A. Ramirez, S. E. Konopelski, A. Elzein, A. Wang, R. J. Supapannachart, H.-L. Lee, C. H. Lim, A. Nace, A. Guo, E. Treffeisen, T. Andl, R. N. Ramirez, R. Murad, S. Offermanns, D. Metzger, P. Chambon, A. D. Widgerow, T.-L. Tuan, A. Mortazavi, R. K. Gupta, B. A. Hamilton, S. E. Millar, P. Seale, W. S. Pear, M. A. Lazar, G. Cotsarelis, Regeneration of fat cells from myofibroblasts during wound healing. Science 355, 748-752 (2017).
34. A. Butler, P. Hoffman, P. Smibert, E. Papalexi, R. Satija, Integrating single-cell transcriptomic data across different conditions, technologies, and species. Nat Biotechnol 36, 411-420 (2018).
35. P. Ramos-Ibeas, F. Sang, Q. Zhu, W. W. C. Tang, S. Withey, D. Klisch, L. Wood, M. Loose, M. A. Surani, R. Alberio, Pluripotency and X chromosome dynamics revealed in pig pre-gastrulating embryos by single cell analysis. Nat Commun 10, 500 (2019).
36. M. S. B. Raredon, T. S. Adams, Y. Suhail, J. C. Schupp, S. Poli, N. Neumark, K. L. Leiby, A. M. Greaney, Y. Yuan, C. Horien, G. Linderman, A. J. Engler, D. J. Boffa, Y. Kluger, I. O. Rosas, A. Levchenko, N. Kaminski, L. E. Niklason, Single-cell connectomic analysis of adult mammalian lungs. Sci Adv 5, eaaw3851 (2019).
37. M. Januszyk, V. W. Wong, K. A. Bhatt, I. N. Vial, J. Paterno, M. T. Longaker, G. C. Gurtner, Mechanical offloading of incisional wounds is associated with transcriptional downregulation of inflammatory pathways in a large animal model. Organogenesis 10, 186-193 (2014).
38. G. C. Gurtner, R. H. Dauskardt, V. W. Wong, K. A. Bhatt, K. Wu, I. N. Vial, K. Padois, J. M. Korman, M. T. Longaker, Improving cutaneous scar formation by controlling the mechanical environment: large animal and phase I studies. Annals of surgery 254, 217-225 (2011).
39. J. L. Hunt, R. Sato, C. R. Baxter, Early tangential excision and immediate mesh autografting of deep dermal hand burns. Annals of surgery 189, 147-151 (1979).
40. D. N. Herndon, R. E. Barrow, R. L. Rutan, T. C. Rutan, M. H. Desai, S. Abston, A comparison of conservative versus early excision. Therapies in severely burned patients. Annals of surgery 209, 547-552; discussion 552-543 (1989).
41. D. C. Adams, M. L. Ramsey, Grafts in dermatologic surgery: review and update on full- and split-thickness skin grafts, free cartilage grafts, and composite grafts. Dermatol Surg 31, 1055-1067 (2005).
42. Y. Bian, C. Sun, X. Zhang, Y. Li, W. Li, X. Lv, J. Li, L. Jiang, J. Li, J. Feng, X. Y. Li, Wound-healing improvement by resurfacing split-thickness skin donor sites with thin split-thickness grafting. Burns: journal of the International Society for Burn Injuries 42, 123-130 (2016).
43. G. Y. Hur, D. K. Seo, J. W. Lee, Contracture of skin graft in human burns: effect of artificial dermis. Burns: journal of the International Society for Burn Injuries 40, 1497-1503 (2014).
44. M. Januszyk, K. Chen, D. Henn, D. S. Foster, M. R. Borrelli, C. A. Bonham, D. Sivaraj, D. Wagh, M. T. Longaker, D. C. Wan, G. C. Gurtner, Characterization of Diabetic and Non-Diabetic Foot Ulcers Using Single-Cell RNA-Sequencing. Micromachines (Basel) 11, (2020).
45. A. Diaz-Papkovich, L. Anderson-Trocme, C. Ben-Eghan, S. Gravel, UMAP reveals cryptic population structure and phenotype heterogeneity in large genomic cohorts. PLoS Genet 15, e1008432 (2019).
46. A. M. Ascensión, S. Fuertes-Álvarez, O. Ibañez-Solé, A. Izeta, M. J. Araúzo-Bravo, Human Dermal Fibroblast Subpopulations Are Conserved across Single-Cell RNA Sequencing Studies. The Journal of investigative dermatology 141, 1735-1744.e1735 (2021).
47. V. Vorstandlechner, M. Laggner, P. Kalinina, W. Haslik, C. Radtke, L. Shaw, B. M. Lichtenberger, E. Tschachler, H. J. Ankersmit, M. Mildner, Deciphering the functional heterogeneity of skin fibroblasts using single-cell RNA sequencing. FASEB journal: official publication of the Federation of American Societies for Experimental Biology 34, 3677-3692 (2020).
48. H. He, H. Suryawanshi, P. Morozov, J. Gay-Mimbrera, E. Del Duca, H. J. Kim, N. Kameyama, Y. Estrada, E. Der, J. G. Krueger, J. Ruano, T. Tuschl, E. Guttman-Yassky, Single-cell transcriptome analysis of human skin identifies novel fibroblast subpopulation and enrichment of immune subsets in atopic dermatitis. J Allergy Clin Immunol 145, 1615-1628 (2020).
49. C. C. Deng, Y. F. Hu, D. H. Zhu, Q. Cheng, J. J. Gu, Q. L. Feng, L. X. Zhang, Y. P. Xu, D. Wang, Z. Rong, B. Yang, Single-cell RNA-seq reveals fibroblast heterogeneity and increased mesenchymal fibroblasts in human fibrotic skin diseases. Nat Commun 12, 3709 (2021).
50. D. R. Zerbino, P. Achuthan, W. Akanni, M. R. Amode, D. Barrell, J. Bhai, K. Billis, C. Cummins, A. Gall, C. G. Girón, Ensembl 2018. Nucleic acids research 46, D754-D761 (2018).
51. A. Dobin, C. A. Davis, F. Schlesinger, J. Drenkow, C. Zaleski, S. Jha, P. Batut, M. Chaisson, T. R. Gingeras, STAR: ultrafast universal RNA-seq aligner. Bioinformatics 29, 15-21 (2013).
52. T. Stuart, A. Butler, P. Hoffman, C. Hafemeister, E. Papalexi, W. M. Mauck III, Y. Hao, M. Stoeckius, P. Smibert, R. Satija, Comprehensive Integration of Single-Cell Data. Cell, (2019).
53. N. Gerstner, T. Kehl, K. Lenhof, A. Müller, C. Mayer, L. Eckhart, N. L. Grammes, C. Diener, M. Hart, O. Hahn, J. Walter, T. Wyss-Coray, E. Meese, A. Keller, H. P. Lenhof, GeneTrail 3: advanced high-throughput enrichment analysis. Nucleic Acids Res 48, W515-w520 (2020).
54. R. C. Russo, C. C. Garcia, M. M. Teixeira, F. A. Amaral, The CXCL8/IL-8 chemokine family and its receptors in inflammatory diseases. Expert Rev Clin Immunol 10, 593-619 (2014).
55. N. Oikonomou, V. Harokopos, J. Zalevsky, C. Valavanis, A. Kotanidou, D. E. Szymkowski, G. Kollias, V. Aidinis, Soluble TNF mediates the transition from pulmonary inflammation to fibrosis. PLOS One 1, e108 (2006).
56. J. J. Saucerman, P. M. Tan, K. S. Buchholz, A. D. McCulloch, J. H. Omens, Mechanical regulation of gene expression in cardiac myocytes and fibroblasts. Nature Reviews Cardiology 16, 361-378 (2019).
57. D. J. Tschumperlin, G. Ligresti, M. B. Hilscher, V. H. Shah, Mechanosensing and fibrosis. J Clin Invest 128, 74-84 (2018).
58. S. Adams, L. M. Wuescher, R. Worth, E. Yildirim-Ayan, Mechano-Immunomodulation: Mechanoresponsive Changes in Macrophage Activity and Polarization. Ann Biomed Eng 47, 2213-2231 (2019).
59. B. Aldeiri, U. Roostalu, A. Albertini, J. Wong, A. Morabito, G. Cossu, Transgelin-expressing myofibroblasts orchestrate ventral midline closure through TGFβ signalling. Development 144, 3336-3348 (2017).
60. J. Park, J. Park, J. Jeong, K. H. Cho, I. Choi, J. Kim, Identification of tetranectin as adipogenic serum protein. Biochem Biophys Res Commun 460, 583-588 (2015).
61. T. Satoh, K. Nakagawa, F. Sugihara, R. Kuwahara, M. Ashihara, F. Yamane, Y. Minowa, K. Fukushima, I. Ebina, Y. Yoshioka, A. Kumanogoh, S. Akira, Identification of an atypical monocyte and committed progenitor involved in fibrosis. Nature 541, 96-101 (2017).
62. P. M. Tang, D. J. Nikolic-Paterson, H. Y. Lan, Macrophages: versatile players in renal inflammation and fibrosis. Nat Rev Nephrol 15, 144-158 (2019).
63. X. M. Meng, D. J. Nikolic-Paterson, H. Y. Lan, Inflammatory processes in renal fibrosis. Nat Rev Nephrol 10, 493-503 (2014).
64. D. Henn, K. Chen, T. Fehlmann, A. A. Trotsyuk, D. Sivaraj, Z. N. Maan, C. A. Bonham, Jr., J. A. Barrera, C. J. Mays, A. H. Greco, S. E. Moortgat Illouz, J. Q. Lin, S. R. Steele, D. S. Foster, J. Padmanabhan, A. Momeni, D. Nguyen, D. C. Wan, U. Kneser, M. Januszyk, A. Keller, M. T. Longaker, G. C. Gurtner, Xenogeneic skin transplantation promotes angiogenesis and tissue regeneration through activated Trem2(+) macrophages. Sci Adv 7, eabi4528 (2021).
65. V. M. Golubovskaya, Targeting FAK in human cancer: from finding to first clinical trials. Front Biosci (Landmark Ed) 19, 687-706 (2014).
66. F. J. Sulzmaier, C. Jean, D. D. Schlaepfer, FAK in cancer: mechanistic findings and clinical applications. Nat Rev Cancer 14, 598-610 (2014).
67. W. G. Roberts, E. Ung, P. Whalen, B. Cooper, C. Hulford, C. Autry, D. Richter, E. Emerson, J. Lin, J. Kath, K. Coleman, L. Yao, L. Martinez-Alsina, M. Lorenzen, M. Berliner, M. Luzzio, N. Patel, E. Schmitt, S. LaGreca, J. Jani, M. Wessel, E. Marr, M. Griffor, F. Vajdos, Antitumor activity and pharmacology of a selective focal adhesion kinase inhibitor, PF-562,271. Cancer Res 68, 1935-1944 (2008).
68. R. Fearmonti, J. Bond, D. Erdmann, H. Levinson, A review of scar scales and scar measuring devices. Eplasty 10, e43 (2010).
69. P. Ortiz-Montero, A. Londoño-Vallejo, J. P. Vernot, Senescence-associated IL-6 and IL-8 cytokines induce a self- and cross-reinforced senescence/inflammatory milieu strengthening tumorigenic capabilities in the MCF-7 breast cancer cell line. Cell Commun Signal 15, 17 (2017).
70. C. A. Feghali, T. M. Wright, Cytokines in acute and chronic inflammation. Front Biosci 2, d12-26 (1997).
71. A. M. Tager, R. L. Kradin, P. LaCamera, S. D. Bercury, G. S. Campanella, C. P. Leary, V. Polosukhin, L. H. Zhao, H. Sakamoto, T. S. Blackwell, A. D. Luster, Inhibition of pulmonary fibrosis by the chemokine IP-10/CXCL10. Am J Respir Cell Mol Biol 31, 395-404 (2004).
72. S. D. Oldroyd, G. L. Thomas, G. Gabbiani, A. M. El Nahas, Interferon-gamma inhibits experimental renal fibrosis. Kidney Int 56, 2116-2127 (1999).
73. G. Raghu, K. K. Brown, W. Z. Bradford, K. Starko, P. W. Noble, D. A. Schwartz, T. E. King, Jr., A placebo-controlled trial of interferon gamma-1b in patients with idiopathic pulmonary fibrosis. N Engl J Med 350, 125-133 (2004).
74. T. Poynard, J. McHutchison, M. Manns, C. Trepo, K. Lindsay, Z. Goodman, M. H. Ling, J. Albrecht, Impact of pegylated interferon alfa-2b and ribavirin on liver fibrosis in patients with chronic hepatitis C. Gastroenterology 122, 1303-1313 (2002).
75. B. A. Croker, D. L. Krebs, J. G. Zhang, S. Wormald, T. A. Willson, E. G. Stanley, L. Robb, C. J. Greenhalgh, I. Forster, B. E. Clausen, N. A. Nicola, D. Metcalf, D. J. Hilton, A. W. Roberts, W. S. Alexander, SOCS3 negatively regulates IL-6 signaling in vivo. Nat Immunol 4, 540-545 (2003).
76. J. Wu, A. H. Lewis, J. Grandl, Touch, Tension, and Transduction—The Function and Regulation of Piezo Ion Channels. Trends Biochem Sci 42, 57-71 (2017).
77. J. Padmanabhan, M. J. Augelli, B. Cheung, E. R. Kinser, B. Cleary, P. Kumar, R. Wang, A. J. Sawyer, R. Li, U. D. Schwarz, J. Schroers, T. R. Kyriakides, Regulation of cell-cell fusion by nanotopography. Sci Rep 6, 33277 (2016).
78. K. Chen, D. Henn, D. Sivaraj, C. A. Bonham, M. Griffin, H. Choi Kussie, J. Padmanabhan, A. A. Trotsyuk, D. C. Wan, M. Januszyk, M. T. Longaker, G. C. Gurtner, Mechanical Strain Drives Myeloid Cell Differentiation Toward Pro-Inflammatory Subpopulations. Advances in wound care, (2021).
79. R. Agha, R. Ogawa, G. Pietramaggiori, D. P. Orgill, A review of the role of mechanical forces in cutaneous wound healing. J Surg Res 171, 700-708 (2011).
80. V. Bergen, M. Lange, S. Peidli, F. A. Wolf, F. J. Theis, Generalizing RNA velocity to transient cell states through dynamical modeling. Nat Biotechnol, (2020).
81. M. Lange, V. Bergen, M. Klein, M. Setty, B. Reuter, M. Bakhti, H. Lickert, M. Ansari, J. Schniering, H. B. Schiller, D. Pe'er, F. J. Theis, CellRank for directed single-cell fate mapping. bioRxiv, 2020.2010.2019.345983 (2020).
82. A. Jeschke, M. Bonitz, M. Simon, S. Peters, W. Baum, G. Schett, W. Ruether, A. Niemeier, T. Schinke, M. Amling, Deficiency of Thrombospondin-4 in Mice Does Not Affect Skeletal Growth or Bone Mass Acquisition, but Causes a Transient Reduction of Articular Cartilage Thickness. PLOS One 10, e0144272 (2015).
83. S. A. Wong, D. P. Hu, J. Slocum, C. Lam, M. Nguyen, T. Miclau, R. S. Marcucio, C. S. Bahney, Chondrocyte-to-osteoblast transformation in mandibular fracture repair. J Orthop Res, (2020).
84. P. Smeriglio, F. C. Grandi, S. E. B. Taylor, A. Zalc, N. Bhutani, TET1 Directs Chondrogenic Differentiation by Regulating SOX9 Dependent Activation of Col2a1 and Acan In Vitro. JBMR Plus 4, e10383 (2020).
85. B. Sid, H. Sartelet, G. Bellon, H. El Btaouri, G. Rath, N. Delorme, B. Haye, L. Martiny, Thrombospondin 1: a multifunctional protein implicated in the regulation of tumor growth. Crit Rev Oncol Hematol 49, 245-258 (2004).
86. M. Mastri, Z. Shah, K. Hsieh, X. Wang, B. Wooldridge, S. Martin, G. Suzuki, T. Lee, Secreted Frizzled-related protein 2 as a target in antifibrotic therapeutic intervention. Am J Physiol Cell Physiol 306, C531-539 (2014).
87. D. Honardoust, M. Varkey, K. Hori, J. Ding, H. A. Shankowsky, E. E. Tredget, Small leucine-rich proteoglycans, decorin and fibromodulin, are reduced in postburn hypertrophic scar. Wound repair and regeneration: official publication of the Wound Healing Society [and] the European Tissue Repair Society 19, 368-378 (2011).
88. R. A. Ignotz, J. Massagué, Type beta transforming growth factor controls the adipogenic differentiation of 3T3 fibroblasts. Proceedings of the National Academy of Sciences of the United States of America 82, 8530-8534 (1985).
89. M. Augsten, C. Hägglöf, E. Olsson, C. Stolz, P. Tsagozis, T. Levchenko, M. J. Frederick, A. Borg, P. Micke, L. Egevad, A. Ostman, CXCL14 is an autocrine growth factor for fibroblasts and acts as a multi-modal stimulator of prostate tumor growth. Proceedings of the National Academy of Sciences of the United States of America 106, 3414-3419 (2009).
90. R. J. Waldemer-Streyer, A. Reyes-Ordoñez, D. Kim, R. Zhang, N. Singh, J. Chen, Cxcl14 depletion accelerates skeletal myogenesis by promoting cell cycle withdrawal. NPJ Regen Med 2, 16017—(2017).
91. N. J. Song, S. Kim, B. H. Jang, S. H. Chang, U. J. Yun, K. M. Park, H. Waki, D. Y. Li, P. Tontonoz, K. W. Park, Small Molecule-Induced Complement Factor D (Adipsin) Promotes Lipid Accumulation and Adipocyte Differentiation. PLOS One 11, e0162228 (2016).
92. Z. H. Huang, C. A. Reardon, T. Mazzone, Endogenous ApoE expression modulates adipocyte triglyceride content and turnover. Diabetes 55, 3394-3402 (2006).
93. R. W. Mahley, T. L. Innerarity, S. C. Rall, Jr., K. H. Weisgraber, Plasma lipoproteins: apolipoprotein structure and function. J Lipid Res 25, 1277-1294 (1984).
94. B. M. Varisco, N. Ambalavanan, J. A. Whitsett, J. S. Hagood, Thy-1 signals through PPARγ to promote lipofibroblast differentiation in the developing lung. Am J Respir Cell Mol Biol 46, 765-772 (2012).
95. W. Qian, N. Li, Q. Cao, J. Fan, Thrombospondin-4 critically controls transforming growth factor β1 induced hypertrophic scar formation. J Cell Physiol 234, 731-739 (2018).
96. V. W. Wong, K. Levi, S. Akaishi, G. Schultz, R. H. Dauskardt, Scar zones: region-specific differences in skin tension may determine incisional scar formation. Plastic and reconstructive surgery 129, 1272-1276 (2012).
97. R. Maiti, L.-C. Gerhardt, Z. S. Lee, R. A. Byers, D. Woods, J. A. Sanz-Herrera, S. E. Franklin, R. Lewis, S. J. Matcher, M. J. Carré, In vivo measurement of skin surface strain and sub-surface layer deformation induced by natural tissue stretching. Journal of the Mechanical Behavior of Biomedical Materials 62, 556-569 (2016).
98. K. Chen, A. Vigliotti, M. Bacca, R. M. McMeeking, V. S. Deshpande, J. W. Holmes, Role of boundary conditions in determining cell alignment in response to stretch. Proceedings of the National Academy of Sciences of the United States of America 115, 986-991 (2018).
99. J. Crawford, K. Nygard, B. S. Gan, D. B. O'Gorman, Periostin induces fibroblast proliferation and myofibroblast persistence in hypertrophic scarring. Exp Dermatol 24, 120-126 (2015).
100. D. Sivaraj, K. Chen, A. Chattopadhyay, D. Henn, W. Wu, C. Noishiki, N. J. Magbual, S. Mittal, A. M. Mermin-Bunnell, C. A. Bonham, A. A. Trotsyuk, J. A. Barrera, J. Padmanabhan, M. Januszyk, G. C. Gurtner, Hydrogel Scaffolds to Deliver Cell Therapies for Wound Healing. Front Bioeng Biotechnol 9, 660145 (2021).
101. T. Xie, Y. Wang, N. Deng, G. Huang, F. Taghavifar, Y. Geng, N. Liu, V. Kulur, C. Yao, P. Chen, Z. Liu, B. Stripp, J. Tang, J. Liang, P. W. Noble, D. Jiang, Single-Cell Deconvolution of Fibroblast Heterogeneity in Mouse Pulmonary Fibrosis. Cell Rep 22, 3625-3640 (2018).
102. S. Mahmoudi, E. Mancini, L. Xu, A. Moore, F. Jahanbani, K. Hebestreit, R. Srinivasan, X. Li, K. Devarajan, L. Prélot, C. E. Ang, Y. Shibuya, B. A. Benayoun, A. L. S. Chang, M. Wernig, J. Wysocka, M. T. Longaker, M. P. Snyder, A. Brunet, Heterogeneity in old fibroblasts is linked to variability in reprogramming and wound healing. Nature 574, 553-558 (2019).
103. K. Sudo, M. Kanno, K. Miharada, S. Ogawa, T. Hiroyama, K. Saijo, Y. Nakamura, Mesenchymal progenitors able to differentiate into osteogenic, chondrogenic, and/or adipogenic cells in vitro are present in most primary fibroblast-like cell populations. Stem Cells 25, 1610-1617 (2007).
104. R. McBeath, D. M. Pirone, C. M. Nelson, K. Bhadriraju, C. S. Chen, Cell shape, cytoskeletal tension, and RhoA regulate stem cell lineage commitment. Dev Cell 6, 483-495 (2004).
105. S. Y. Jeong, D. H. Kim, J. Ha, H. J. Jin, S. J. Kwon, J. W. Chang, S. J. Choi, W. Oh, Y. S. Yang, G. Kim, J. S. Kim, J. R. Yoon, D. H. Cho, H. B. Jeon, Thrombospondin-2 secreted by human umbilical cord blood-derived mesenchymal stem cells promotes chondrogenic differentiation. Stem Cells 31, 2136-2148 (2013).
106. H. S. Shitaye, S. P. Terkhorn, J. A. Combs, K. D. Hankenson, Thrombospondin-2 is an endogenous adipocyte inhibitor. Matrix Biol 29, 549-556 (2010).
107. P. Martin, Wound healing—aiming for perfect skin regeneration. Science 276, 75-81 (1997).
108. R. Edgar, M. Domrachev, A. E. Lash, Gene Expression Omnibus: NCBI gene expression and hybridization array data repository. Nucleic Acids Res 30, 207-210 (2002).
109. J. S. Bredfeldt, Y. Liu, C. A. Pehlke, M. W. Conklin, J. M. Szulczewski, D. R. Inman, P. J. Keely, R. D. Nowak, T. R. Mackie, K. W. Eliceiri, Computational segmentation of collagen fibers from second-harmonic generation images of breast cancer. J Biomed Opt 19, 16007-16007 (2014).
110. G. M. Fomovsky, J. W. Holmes, Evolution of scar structure, mechanics, and ventricular function after myocardial infarction in the rat. American journal of physiology. Heart and circulatory physiology 298, H221-228 (2010).
111. H. S. Ryu, Y. H. Joo, S. O. Kim, K. C. Park, S. W. Youn, Influence of age and regional differences on skin elasticity as measured by the Cutometer. Skin research and technology: official journal of International Society for Bioengineering and the Skin (ISBS) [and] International Society for Digital Imaging of Skin (ISDIS) [and] International Society for Skin Imaging (ISSI) 14, 354-358 (2008).
112. S. S. Fong, L. K. Hung, J. C. Cheng, The cutometer and ultrasonography in the assessment of postburn hypertrophic scar—a preliminary study. Burns: journal of the International Society for Burn Injuries 23 Suppl 1, S12-18 (1997).
113. Y. Liu, A. Keikhosravi, G. S. Mehta, C. R. Drifka, K. W. Eliceiri, Methods for Quantifying Fibrillar Collagen Alignment. Methods Mol Biol 1627, 429-451 (2017).
114. D. Osorio, J. J. Cai, Systematic determination of the mitochondrial proportion in human and mice tissues for single-cell RNA sequencing data quality control. Bioinformatics, (2020).
115. E. Becht, L. McInnes, J. Healy, C.-A. Dutertre, I. W. Kwok, L. G. Ng, F. Ginhoux, E. W. Newell, Dimensionality reduction for visualizing single-cell data using UMAP. Nature biotechnology 37, 38 (2019).
116. R. K. Auerbach, B. Chen, A. J. Butte, Relating genes to function: identifying enriched transcription factors using the ENCODE ChIP-Seq significance tool. Bioinformatics 29, 1922-1924 (2013). 0.5
117. R. A. Horn, V. V. Sergeichuk, Congruences of a square matrix and its transpose. Linear Algebra and its Applications 389, 347-353 (2004).

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. Moreover, nothing disclosed herein is intended to be dedicated to the public regardless of whether such disclosure is explicitly recited in the claims.

The scope of the present invention, therefore, is not intended to be limited to the exemplary embodiments shown and described herein. Rather, the scope and spirit of present invention is embodied by the appended claims. In the claims, 35 U.S.C. § 112(f) or 35 U.S.C. § 112(6) is expressly defined as being invoked for a limitation in the claim only when the exact phrase “means for” or the exact phrase “step for” is recited at the beginning of such limitation in the claim; if such exact phrase is not used in a limitation in the claim, then 35 U.S.C. § 112 (f) or 35 U.S.C. § 112(6) is not invoked.

1. A method of treating a wound of a subject, the method comprising:
applying a skin graft to the wound in combination with a mechanotransduction blocker to treat the wound of the subject.

2. The method according to claim 1, wherein the skin graft is a split-thickness skin graft.

3. The method according to claim 1, wherein the wound is a deep injury wound.

4. The method according to claim 1, wherein the deep injury wound is a burn wound.

5. The method according to claim 1, wherein the deep injury wound is a traumatic wound.

6. The method according to claim 1, wherein the skin graft is applied to the wound before the mechanotransduction blocker.

7. The method according to claim 1, wherein the mechanotransduction blocker comprises a pharmacological mechanotransduction blocker.

8. The method according to claim 1, wherein the pharmacological mechanotransduction blocker comprises a focal adhesion kinase inhibitor.

9. The method according to claim 1, wherein the mechanotransduction blocker is administered in a sustained release formulation to the wound.

10. The method according to claim 8, wherein the sustained release formulation comprises a gel formulation.

11. The method according to claim 10, wherein the gel formulation comprises a hydrogel.

12. The method according to claim 11, wherein hydrogel comprises a biodegradable pullulan-based hydrogel.

13. The method according to claim 1, wherein the method promotes healing of the wound.

14. The method according to claim 1, wherein the method reduces fibrosis.

15. The method according to claim 1, wherein the method reduces contracture.

16. The method according to claim 1, wherein the method mitigates scar formation.

17. The method according to claim 1, wherein the method restores collagen architecture.

18. The method according to claim 1, wherein the method improves graft biomechanical properties.

19. The method according to claim 1, wherein the subject is mammal.

20. The method according to claim 19, wherein the mammal is a human.

21-30. (canceled)
